SEQUENCE LISTING (13 sequences)

SEQ ID NO: 1 - protein, 11 aa, Artificial sequence
  membrane-permeable peptide
GRKKRRQRRRG

SEQ ID NO: 2 - protein, 10 aa, Artificial sequence
  membrane-permeable peptide
CHHRRRRHHC

SEQ ID NO: 3 - protein, 13 aa, Artificial sequence
  membrane-permeable peptide
GRKKRRQRRRPPQ

SEQ ID NO: 4 - protein, 5 aa, Artificial sequence
  membrane-permeable peptide
CHHRR

SEQ ID NO: 5 - protein, 8 aa, Artificial sequence
  membrane-permeable peptide
HHRRRRHH

SEQ ID NO: 6 - protein, 8 aa, Artificial sequence
  membrane-permeable peptide
HHHHRRRR

SEQ ID NO: 7 - protein, 8 aa, Artificial sequence
  membrane-permeable peptide
RRRRHHHH

SEQ ID NO: 8 - protein, 16 aa, Artificial sequence
  membrane-permeable peptide
RQIKIWFQNRRMKWKK

SEQ ID NO: 9 - protein, 19 aa, Artificial sequence
  membrane-permeable peptide
GWTLNSAGYLLGKINLKAL

SEQ ID NO: 10 - protein, 21 aa, Artificial sequence
  membrane-permeable peptide
KETWWETWWTEWSQPKKKRKV

SEQ ID NO: 11 - protein, 18 aa, Artificial sequence
  membrane-permeable peptide
LLIILRRRIRKQAHAHSK

SEQ ID NO: 12 - RNA, 21 nt, Artificial Sequence
  hSOD1 siRNA
caaagaugcuguggccgaugu

SEQ ID NO: 13 - DNA, 21 bp, Artificial Sequence
  control siRNA
auccgcgcgauaguacguatt

(19) United States
(12) Patent Application Publication
(10) Pub. No.: US 2022/0241422 A1
(43) Pub. Date: Aug. 4, 2022

(54) DRUG DELIVERY COMPOSITION AND PHARMACEUTICAL COMPOSITION

(71) Applicants: Nihon University, Chiyoda-ku, Tokyo (JP); Shizuoka Prefectural University Corporation, Shizuoka-shi, Shizuoka (JP)

(72) Inventors: Takanori KANAZAWA, Shizuoka-shi, Shizuoka (JP); Yasuhiro KOSUGE, Chiyoda-ku, Tokyo (JP); Hiroko MIYAGISHI, Chiyoda-ku, Tokyo (JP); Naoto SUZUKI, Chiyoda-ku, Tokyo (JP)

(21) Appl. No.: 17/620,192

(22) PCT Filed: May 7, 2020
(86) PCT No.: PCT/JP2020/018512

§ 371 Date: Dec. 17, 2021

(30) Foreign Application Priority Data
Jun. 21, 2019 (JP) 2019-115688

(51) Int. Cl. A61K 47/34; A61K 45/06; A61P 25/04; A61K 9/00

(52) U.S. Cl. CPC A61K 47/34; A61K 9/0043; A61P 25/04; A61K 45/06

(57) ABSTRACT

A drug delivery composition for delivering a drug to the spinal cord, the drug delivery composition containing a block copolymer having a polyethylene glycol segment and a hydrophobic polyester segment linked together, and a membrane-permeable peptide, in which the drug delivery composition is administered nasally. A pharmaceutical composition for treating a spinal cord disease, the pharmaceutical composition containing the above-mentioned drug delivery composition and a drug for treatment of a spinal cord disease, in which the pharmaceutical composition is administered nasally.

### TECHNICAL FIELD

The present invention relates to a drug delivery composition and a pharmaceutical composition. The drug delivery composition of the present invention is administered nasally and is used to deliver a drug to the spinal cord.

Priority is claimed on Japanese Patent Application No. 2019-115688, filed Jun. 21, 2019, the content of which is incorporated herein by reference.

### BACKGROUND ART

Drug delivery to the spinal cord mediated by systemically circulating blood through oral administration or intravenous administration is significantly restricted by the blood-brain barrier and blood-cerebrospinal fluid barrier, and therefore, it is difficult to increase the drug concentration in the spinal cord tissue to the therapeutic range. Furthermore, since a large amount of drug is to be administered in order to raise the level to the therapeutic range, there is concern that side effects on peripheral tissues may occur via the systemically circulating blood. Intrathecal administration can selectively deliver a drug to the spinal cord tissue; however, it is difficult to plan the administration because the administration requires skill and is highly invasive while imposing a large burden on the patient.

On the other hand, development of drug delivery technologies is in progress as technologies for delivering a drug to a diseased site. For example, a nucleic acid delivery composition containing a block copolymer composed of a Methoxy Polyethylene Glycol (MPEG) segment and a Poly(ε-caprolactone) (PCL) segment and a peptide including a lipid-soluble group, the peptide being composed of 10 amino acids including arginine and histidine, has been reported (Patent Document 1).

### CITATION LIST

### Patent Document

### Patent Document 1

PCT International Publication No. WO 2019/013255

### SUMMARY OF INVENTION

### Technical Problem

There is a demand for the development of a technology capable of delivering a drug to the spinal cord by a simple method that is less invasive. However, it has not been verified whether the nucleic acid delivery composition as described in Patent Document 1 can deliver a drug to the spinal cord.

Thus, it is an object of the present invention to provide a drug delivery composition capable of delivering a drug to the spinal cord by a simple method with low invasiveness, and a pharmaceutical composition containing the drug delivery composition.

### Solution to Problem

The present invention includes the following embodiments.

[1] A drug delivery composition for delivering a drug to the spinal cord, the drug delivery composition containing:
a block copolymer having a polyethylene glycol segment and a hydrophobic polyester segment linked together; and
a membrane-permeable peptide,
in which the drug delivery composition is administered nasally.

[2] The drug delivery composition according to [1], in which the drug is a drug for treatment of a spinal cord disease.

[3] The drug delivery composition according to [2], in which the spinal cord disease is selected from the group consisting of amyotrophic lateral sclerosis, spinocerebellar degeneration, spinal muscular atrophy, primary lateral sclerosis, spinobulbar muscular atrophy, chronic pain, and spinal cord injury.

[4] The drug delivery composition according to any one of [1] to [3], in which the membrane-permeable peptide is bonded to an end of the hydrophobic polyester segment.

[5] The drug delivery composition according to any one of [1] to [3], in which a lipid-soluble group is bonded to the membrane-permeable peptide directly or through a linking group.

[6] The drug delivery composition according to [5], in which the lipid-soluble group is selected from the group consisting of an alkyl group having 4 to 30 carbon atoms which may have a substituent, an alkenyl group having 4 to 30 carbon atoms which may have a substituent, and an aralkyl group having 7 to 30 carbon atoms which may have a substituent.

[7] The drug delivery composition according to any one of [1] to [6], in which the block copolymer and the membrane-permeable peptide form micelles.

[8] A pharmaceutical composition for treating a spinal cord disease, the pharmaceutical composition containing:
the drug delivery composition according to any one of [1] to [7]; and
a drug for treatment of a spinal cord disease,
in which the pharmaceutical composition is administered nasally.

[9] The pharmaceutical composition according to [8], in which the spinal cord disease is selected from the group consisting of amyotrophic lateral sclerosis, spinocerebellar degeneration, spinal muscular atrophy, primary lateral sclerosis, spinobulbar muscular atrophy, chronic pain, and spinal cord injury.

### Advantageous Effects of Invention

According to the present invention, a drug delivery composition capable of delivering a drug to the spinal cord by a simple method with low invasiveness, and a pharmaceutical composition containing the drug delivery composition are provided.

### BRIEF DESCRIPTION OF DRAWINGS

FIG. 1A and FIG. 1B show the results of a nasal administration test for RI-labeled dextran/PEG-PCL-Tat. FIG. 1A shows the efficiency of distribution of RI-labeled dextran into the cerebrum.

FIG. 1B shows the efficiency of distribution of RI-labeled dextran into the spinal cord.

FIG. 2A shows the survival time obtained when N-acetylcysteine (NAC) was administered orally to ALS model mice.

FIG. 2B shows the progression of motor dysfunction occurring when N-acetylcysteine (NAC) was administered orally to ALS model mice.

FIG. 3 shows an outline of a method for a nasal administration test of an ALS therapeutic drug in ALS model mice.

FIG. 4A shows the progression of motor dysfunction occurring when NAC was administered nasally to ALS model mice.

FIG. 4B shows the expression of SMI-32, which is a marker for motor neurons, occurring when NAC was administered nasally to ALS model mice.

FIG. 5 shows the progression of motor dysfunction occurring when a cyclosporine A/PEG-PCL-Tat complex was administered nasally to ALS model mice.

FIG. 6A and FIG. 6B show the results of allodynia responses to tactile stimuli obtained when a NAC/PEG-PCL-Tat complex was administered nasally to neuropathic pain model mice. FIG. 6A shows the results for the unligated side hind limb (left limb).

FIG. 6B shows the results for the ligated side hind limb (right limb).

FIG. 7 shows the results for a nasal administration test of an RI-labeled dextran/PEG-PCL/peptide complex.

### DESCRIPTION OF EMBODIMENTS

### Definitions

In the present specification, the term “n-” means normal, “i-” means iso, “s-” means secondary, and “t-” means tertiary.

The term “derived group” means a group obtained by removing a hydrogen atom at any position from a target molecule.

The phrase “may have a substituent” means that it is unsubstituted or substituted with at least one substituent. The substituent includes both a case where a hydrogen atom (—H) is substituted with a monovalent group, and a case where a methylene group —CH₂—) is substituted with a divalent group.

A “halogen atom” means a fluorine atom, a chlorine atom, a bromine atom, or an iodine atom.

Unless particularly stated otherwise, an “alkyl group” is meant to include linear, branched, and cyclic monovalent saturated hydrocarbon groups. Specific exemplary examples of the alkyl group include a methyl group, an ethyl group, an n-propyl group, an i-propyl group, an n-butyl group, an i-butyl group, an s-butyl group, a t-butyl group, an n-pentyl group, a cyclopentyl group, an n-hexyl group, a cyclohexyl group, a cyclohexylmethyl group, a cyclohexylethyl group, an n-octyl group, an n-nonyl group, an n-decyl group, an n-undecyl group, an n-dodecyl group, an n-tridecyl group, an n-tetradecyl group, an n-pentadecyl group, an n-hexadecyl group, an n-heptadecyl group, an n-octadecyl group, an n-nonadecyl group, an n-eicosyl group, an isooctyl group, an isodecyl group, an isododecyl group, an isotetradecyl group, an isohexadecyl group, an isooctadecyl group, a t-octyl group, a t-decyl group, a t-dodecyl group, a t-tetradecyl group, a t-hexadecyl group, and a t-octadecyl group.

Specific examples of an alkyl group having a to b carbon atoms as described in the present specification include those specific examples of the alkyl group respectively having their number of carbon atoms in the specified ranges. The same applies to the following groups.

An “alkenyl group” is a linear, branched, or cyclic unsaturated hydrocarbon group having a carbon-carbon double bond at least at any one site, and unless particularly stated otherwise, the alkenyl group is meant to include linear, branched, and cyclic groups. Specific exemplary examples of the alkenyl group include a 1-propenyl group, a 1-butenyl group, a 2-methyl-2-butenyl group, a 2-methyl-1,3-butadienyl group, a 1-octenyl group, a 1-decenyl group, a 1-dodecenyl group, a 1-tetradecenyl group, a 1-hexadecenyl group, a 1-cyclohexenyl group, a 3-cyclohexenyl group, a 1-octadecenyl group, a cis-9-octadecenyl group, and a 9-hexadecenyl group.

An “aryl group” includes a carbocyclic aryl group and a heterocyclic aryl group.

Examples of the carbocyclic aryl group include a phenyl group and a naphthyl group.

A heterocyclic aryl group means a monocyclic or fused ring-based aryl group containing 1 to 5 heteroatoms selected from the group consisting of a nitrogen atom, an oxygen atom, and a sulfur atom among the atoms constituting the ring. Specific exemplary examples of the heterocyclic aryl group include a pyridyl group, a pyrimidinyl group, a quinolyl group, a quinazolinyl group, a naphthyridinyl group, a furyl group, a pyrrolyl group, an imidazolyl group, a pyrazolyl group, an oxazolyl group, an isoxazolyl group, a triazolyl group, a thienyl group, a thiazolyl group, an isothiazolyl group, an indolyl group, a benzofuranyl group, a benzothienyl group, and an imidazopyridyl group.

An “aralkyl group” is an alkyl group in which a hydrogen atom at any one site is substituted with a carbocyclic aryl group. Unless particularly stated otherwise, the alkyl group in the aralkyl group is meant to include linear, branched, and cyclic groups. Specific exemplary examples of the aralkyl group include a benzyl group, a 1-phenylethyl group, a 2-phenylethyl group, a 4-phenylbutyl group, a 3-phenylbutyl group, a 5-phenylpentyl group, a 6-phenylhexyl group, and an 8-phenyloctyl group. Preferred examples thereof include a 4-phenylbutyl group, a 5-phenylpentyl group, a 6-phenylhexyl group, and an 8-phenyloctyl group.

An “alkoxy group” means a group in which an oxy group is bonded to the above-described alkyl group, and unless particularly stated otherwise, the alkoxy group is meant to include linear, branched, and cyclic groups. Specific exemplary examples of the alkoxy group include a methoxy group, an n-propoxy group, a cyclopropylmethyloxy group, an n-hexyloxy group, an isopropoxy group, an s-butoxy group, a cyclohexyloxy group, a t-butoxy group, and an n-octyloxy group.

An “alkenyloxy group” means a group in which an alkenyl group is bonded to an oxy group, and unless particularly stated otherwise, the alkenyl group is meant to include linear, branched, and cyclic groups. Specific exemplary examples of the alkenyloxy group include a 1-propenyloxy group, a 1-butenyloxy group, a 2-methyl-2-butenyloxy group, a 2-methyl-1,3-butadienyloxy group, a 1-octenyloxy group, a 1-decenyloxy group, a 1-cyclohexenyloxy group, and a 3-cyclohexenyloxy group.

An “aralkyloxy group” means a group in which an aralkyl group is bonded to an oxy group. Unless particularly stated otherwise, the alkyl group in the aralkyloxy group is meant to include linear, branched, and cyclic groups. Specific exemplary examples of the aralkyloxy group include a benzyloxy group and a phenethyloxy group.

An “aryloxy group” means a group in which an aryl group is bonded to an oxy group, the aryloxy group is, for example, a carbocyclic aryloxy group or a heterocyclic aryloxy group, and specific examples thereof include a phenoxy group, a naphthyloxy group, and a pyridyloxy group.

An “alkylene group” is a divalent group obtained by removing a hydrogen atom at any position from an alkyl group, and unless particularly stated otherwise, the alkylene group is meant to include linear, branched, and cyclic groups. Specific exemplary examples of the alkylene group include a methylene group, an ethylene group, a propane-1,3-diyl group, a propane-1,2-diyl group, a propane-1,1-diyl group, a propane-2,2-diyl group, a 2,2-dimethyl-propane-1,3-diyl group, a hexane-1,6-diyl group, a 3-methylbutane-1,2-diyl group, and a cyclopropane-1,2-diyl group.

An “alkylthio group” means a group in which an alkyl group is bonded to a thio group, and unless particularly stated otherwise, the alkylthio group is meant to include linear, branched, and cyclic groups. Specific exemplary examples of the alkylthio group include a methylthio group, an ethylthio group, an isopropylthio group, a cyclopropylmethylthio group, a cyclopentylthio group, an n-hexylthio group, and a cyclohexylthio group.

An “aralkylthio group” means a group in which an aralkyl group is bonded to a thio group. Unless particularly stated otherwise, the alkyl group in the aralkyloxy group is meant to include linear, branched, and cyclic groups. Specific exemplary examples of the aralkylthio group include a benzylthio group and a phenethylthio group.

An “arylthio group” means a group in which an aryl group is bonded to a thio group, the arylthio group is, for example, a carbocyclic arylthio group or a heterocyclic arylthio group, and specific examples thereof include a phenylthio group, a naphthylthio group, and a pyridylthio group.

An “alkylsulfinyl group” means a group in which an alkyl group is bonded to a sulfinyl group, and unless particularly stated otherwise, the alkylsulfinyl group includes linear, branched, and cyclic groups. Specific exemplary examples of the alkylfulfinyl group include a methylsulfinyl group, an isopropylsulfinyl group, and a cyclohexylsulfinyl group.

An “aralkylsulfinyl group” means a group in which an aralkyl group is bonded to a sulfinyl group. Unless particularly stated otherwise, the alkyl group in the aralkylsulfinyl group is meant to include linear, branched, and cyclic groups. Specific exemplary examples of the aralkylsulfinyl group include a benzylsulfinyl group and a phenethylsulfinyl group.

An “arylsulfinyl group” means a group in which an aryl group is bonded to a sulfinyl group, the arylsulfinyl group is, for example, a carbocyclic arylsulfinyl group or a heterocyclic arylsulfinyl group, and specific examples thereof include a phenylsulfinyl group, a naphthylsulfinyl group, and a pyridylsulfinyl group.

An “alkylsulfonyl group” means a group in which an alkyl group is bonded to a sulfonyl group, and unless particularly stated otherwise, the alkylsulfonyl group includes linear, branched, and cyclic groups. Specific exemplary examples of the alkylsulfonyl group include a methylsulfonyl group and an isopropylsulfonyl group.

An “aralkylsulfonyl group” means a group in which an aralkyl group is bonded to a sulfonyl group. Unless particularly stated otherwise, the alkyl group in the aralkylsulfonyl group is meant to include linear, branched, and cyclic groups. Specific exemplary examples of the aralkylsulfonyl group include a benzylsulfonyl group and a phenethylsulfonyl group.

An “arylsulfonyl group” means a group in which an aryl group is bonded to a sulfonyl group, the arylsulfonyl group is, for example, a carbocyclic arylsulfonyl group or a heterocyclic arylsulfonyl group, and specific examples thereof include a phenylsulfonyl group, a naphthylsulfonyl group, and a pyridylsulfonyl group.

A “monoalkylamino group” means a group in which one alkyl group is bonded to an amino group, and unless particularly stated otherwise, the monoalkylamino group includes linear, branched, and cyclic groups. Specific exemplary examples of the monoalkylamino group include a methylamino group, an isopropyl amino group, a neopentylamino group, an n-hexylamino group, a cyclohexylamino group, and an n-octylamino group.

A “dialkylamino group” means a group in which two identical or different alkyl groups are bonded to an amino group. Unless particularly stated otherwise, the alkyl group in the dialkylamino group is meant to include linear, branched, and cyclic groups. Specific exemplary examples of the dialkylamino group include a dimethylamino group, a diisopropylamino group, and an N-methyl-N-cyclohexylamino group.

A “cyclic amino group” is a group obtained by removing one hydrogen atom bonded to a nitrogen atom from a 3-membered to 11-membered saturated heterocyclic ring containing at least one nitrogen atom as an atom constituting the ring. Specific examples include a morpholino group, a piperazin-1-yl group, a 4-methylpiperazin-1-yl group, a piperidin-1-yl group, and a pyrrolidin-1-yl group.

A “monoarylamino group” means a group in which one aryl group is bonded to an amino group, the monoarylamino group is, for example, a carbocyclic arylamino group or a heterocyclic arylamino group, and specific examples thereof include a phenylamino group, a naphthylamino group, and a pyridylamino group.

A “diarylamino group” means a group in which two identical or different aryl groups are bonded to an amino group, the diarylamino group is, for example, a di(carbocyclic aryl)amino group, a di(heterocyclic aryl)amino group, or an N-(carbocyclic aryl)-N-(heterocyclic aryl)amino group, and specific examples thereof include a diphenylamino group and an N-phenyl-N-pyridylamino group.

An “acyl group” means a group in which a hydrogen atom, an alkyl group, an alkenyl group, an aryl group, or an aralkyl group is bonded to a carbonyl group, and unless particularly stated otherwise, the acyl group is meant to include linear, branched, and cyclic groups. Specific exemplary examples of the acyl group include a formyl group, an acetyl group, a pivaloyl group, a benzoyl group, and a pyridylcarbonyl group.

An “alkoxycarbonyl group” means a group in which an alkoxy group is bonded to a carbonyl group, and unless particularly stated otherwise, the alkoxycarbonyl group is meant to include linear, branched, and cyclic groups. Specific exemplary examples of the alkoxycarbonyl group include a methoxycarbonyl group and a t-butoxycarbonyl group.

An “aralkyloxycarbonyl group” means a group in which an aralkyloxy group is bonded to a carbonyl group, and unless particularly stated otherwise, the aralkyloxycarbonyl group is meant to include linear, branched, and cyclic groups. Specific exemplary examples of the aralkyloxycarbonyl group include a benzyloxycarbonyl group.

An “acyloxy group” means a group in which an acyl group is bonded to an oxy group, and unless particularly stated otherwise, the acyloxy group is meant to include linear, branched, and cyclic groups. Specific exemplary examples of the acyloxy group include a formyloxy group, an acetoxy group, a benzoyloxy group, and a pyridylcarbonyloxy group.

An “alkoxycarbonyloxy group” means a group in which an alkoxycarbonyl group is bonded to an oxy group, and unless particularly stated otherwise, the alkoxycarbonyloxy group is meant to include linear, branched, and cyclic groups. Specific exemplary examples of the alkoxycarbonyloxy group include a methoxycarbonyloxy group and a t-butoxycarbonyloxy group.

An “aralkyloxycarbonyloxy group” means a group in which an aralkyloxycarbonyl group is bonded to an oxy group, and unless particularly stated otherwise, the aralkyloxycarbonyloxy group is meant to include linear, branched, and cyclic groups. Specific exemplary examples of the aralkyloxycarbonyloxy group include a benzyloxycarbonyloxy group.

An “acylamino group” means a group in which an acyl group is bonded to an amino group, and unless particularly stated otherwise, the acylamino group is meant to include linear, branched, and cyclic groups. Specific exemplary examples of the acylamino group include a formylamino group, an acetylamino group, and a benzoylamino group.

An “alkoxycarbonylamino group” means a group in which an alkoxycarbonyl group is bonded to an amino group, and unless particularly stated otherwise, the alkoxycarbonylamino group is meant to include linear, branched, and cyclic groups. Specific exemplary examples of the alkoxycarbonylamino group include a methoxycarbonylamino group and an ethoxycarbonylamino group.

An “aralkyloxycarbonylamino group” means a group in which an aralkyloxycarbonyl group is bonded to an amino group, and unless particularly stated otherwise, the aralkyloxycarbonylamino group is meant to include linear, branched, and cyclic groups. Specific exemplary examples of the aralkyloxycarbonylamino group include a benzyloxycarbonylamino group.

An “alkylsulfonylamino group” means a group in which an alkylsulfonyl group is bonded to an amino group, and unless particularly stated otherwise, the alkylsulfonylamino group is meant to include linear, branched, and cyclic groups. Specific exemplary examples of the alkylsulfonylamino group include a methanesulfonylamino group.

An “arylsulfonylamino group” means a group in which an arylsulfonyl group is bonded to an amino group, the arylsulfonylamino group is, for example, a carbocyclic arylsulfonylamino group or a heterocyclic arylsulfonylamino group, and specific examples thereof include a benzenesulfonylamino group and a pyridylsulfonylamino group.

A carbamoyl group having a substituent means a group in which the monoalkylamino group, the dialkylamino group, the cyclic amino group, the monoarylamino group, or the diarylamino group is bonded to a carbonyl group, and examples thereof include a dimethylcarbamoyl group and a phenylcarbamoyl group.

A sulfamoyl group having a substituent means a group in which the monoalkylamino group, the dialkylamino group, the cyclic amino group, the monoarylamino group, or the diarylamino group is bonded to a sulfonyl group, and examples thereof include a dimethylsulfamoyl group and a phenylsulfamoyl group.

A carbamoyloxy group having a substituent means a group in which the carbamoyl group having a substituent is bonded to an oxy group, and examples thereof include a dimethylcarbamoyloxy group and a phenylcarbamoyloxy group.

A sulfamoylamino group having a substituent means a group in which the sulfamoyl group having a substituent is bonded to the nitrogen atom of an amino group, the monoalkylamino group, or the monoarylamino group, and examples thereof include a dimethylsulfamoylamino group.

A ureido group having a substituent means a group in which the carbamoyl group having a substituent is bonded to the nitrogen atom of an amino group, the monoalkylamino group, or the monoarylamino group, and examples thereof include a trimethylureido group and a 1-methyl-3-phenyl-ureido group.

Examples of a silyl group include a trialkylsilyl group and a monoalkyldiarylsilyl group. Examples of the alkyl group in the silyl group include an alkyl group having 1 to 6 carbon atoms. Specific exemplary examples thereof include a trimethylsilyl group, a triethylsilyl group, a triisopropylsilyl group, a t-butyldimethylsilyl group, and a t-butyldiphenylsilyl group.

The term “peptide” refers to a polymer of amino acids linked by amide bonds. The peptide may be a polymer of natural amino acids, may be a polymer of natural amino acids and unnatural amino acids (chemical analogs, modified derivatives, and the like of natural amino acids), or may be a polymer of unnatural amino acids. Unless particularly stated otherwise, the amino acid sequence is represented by the single-letter code or the three-letter code of the IUPAC-IUB Guidelines, from the N-terminal side toward the C-terminal side.

### Drug Delivery Composition

A first aspect of the present invention is a drug delivery composition for delivering a drug to the spinal cord, the drug delivery composition containing a block copolymer having a polyethylene glycol segment and a hydrophobic polyester segment linked together, and a membrane-permeable peptide, in which the drug delivery composition is administered nasally.

### Drug

The drug delivery composition according to the present embodiment is a drug composition intended for delivering a drug to the spinal cord. The drug is preferably a drug for treatment of a spinal cord disease. As shown in the Examples that will be described later, a drug can be efficiently delivered to the spinal cord by nasally administering the drug delivery composition of the present embodiment.

The term “spinal cord disease” refers to a disease caused by an injury or dysfunction of the spinal cord. Examples of the spinal cord disease include diseases selected from the group consisting of amyotrophic lateral sclerosis (ALS), neuropathic chronic pain, spinal cord injury, spinal muscular atrophy, spinocerebellar degeneration, spinobulbar muscular atrophy, primary lateral sclerosis, and spinal cord tumor.

The drug is not particularly limited as long as it is used for treating spinal cord diseases, and examples include a low-molecular-weight compound, a peptide (a physiologically active peptide, a hormone-like peptide, a cytokine-like peptide, a cyclic peptide, a synthetic peptide, or the like), a protein (an antibody, an enzyme, a nutritional factor, a cytokine, a hormone, or the like), a nucleic acid (a plasmid DNA, a siRNA, an miRNA, an antisense nucleic acid, a shRNA, a pre-miRNA, a pri-miRNA, an mRNA, a decoy nucleic acid, a ribozyme, a DNA aptamer, an RNA aptamer, a DNA enzyme, or the like), and a lipid.

Examples of the drug for treating ALS include an active oxygen scavenger, a CYP1A2 inhibitor, an immunosuppressant, an anti-inflammatory drug, a PGE2 synthase inhibitor, an EP2 receptor inhibitor, a nutritional factor, a vitamin agent glutamate receptor antagonist, a dopamine agonist, a tyrosine kinase inhibitor, a hormone, and a nucleic acid. Specific examples include N-acetylcysteine, cyclosporine A, tacrolimus (FK506), nobiletin, a non-steroidal anti-inflammatory drug, PF-0441848, TG6-10-1, a neurotrophic factor (NGF or NT-1), a brain-derived neurotrophic factor (BDNF or NT-2), hepatocyte growth factor (HGF), vitamin 12, a vitamin B12 derivative, riluzole, perampanel, levodopa, ropinirole, bosutinib, insulin, insulin-like growth factor-1 (IGF-1), erythropoietin, and Tofersen.

Examples of the drug for treating neuropathic chronic pain include an antioxidant, a PGE2 synthase inhibitor, an EP2 receptor inhibitor, an ATP receptor inhibitor, an analgesic, an antidepressant, and an anticonvulsant. Specific examples include N-acetylcysteine, a P2X4 receptor inhibitor (5-BDBD or NP-1815-PX), PPADS, TNP-ATP, a non-steroidal anti-inflammatory drug, acetaminophen, nobiletin, an opioid, tramadol, a tricyclic antidepressant, a serotonin noradrenaline reuptake inhibitor (SNRI), a Ca²⁺ channel α6δ ligand (pregabalin, mirogabalin, or gabapentin), a Na⁺ channel inhibitory action (carbamazepine or lamotrigine), and a GABA-based activating action (sodium valproate or clonazepam).

Examples of the drug for treating a spinal cord injury include an anti-inflammatory agent, an analgesic, an active oxygen scavenger, a neurotrophic factor, a hematopoietic factor, a peptide, and a nucleic acid. Specific examples include adrenocorticosteroid, edaravone, hepatocyte growth factor (HGF), a brain-derived neurotrophic factor (BDNF), and erythropoietin.

Examples of the drug for treating spinal muscular atrophy include an antisense nucleic acid, a splicing modifier, and a siRNA. Specific examples include risdiplam and nusinersen.

Examples of the drug for treating spinocerebellar degeneration include a thyrotropin-releasing hormone (TRH) and a TRH derivative. Specific examples include mRNAs expressing Hirtonin, Ceredist, Bognin, taltirelin, protirelin, mexiletine hydrochloride, acetazolamide, and TRH.

Examples of the drug for treating spinobulbar muscular atrophy include a luteinizing hormone stimulating hormone (LHRH) analog, a heat shock protein (Hsp70) inducer, a ubiquitin-proteasome-based (UPS) activator, and a histone deacetylase (HDAC) inhibitor. Specific examples include leuprorelin, geranylgeranylacetone (GGA), and 17-allylamino-17-demethoxygeldanamycin (17-AAG).

Examples of the drug for treating primary lateral sclerosis include a muscle relaxant. Specific examples include baclofen and dantrolene.

Examples of the drug for treating a spinal cord tumor include an anticancer agent, an analgesic, an anti-inflammatory agent, an antibody, and a nucleic acid.

### Block Copolymer

The drug delivery composition according to the present embodiment contains a block copolymer having a polyethylene glycol segment and a hydrophobic polyester segment linked together.

### Polyethylene Glycol Segment

A polyethylene glycol segment is a segment including a polyethylene glycol chain having a repeating structure of an ethyleneoxy group (—CH₂CH₂O—) unit. The degree of polymerization of the polyethylene glycol segment is, for example, 5 to 12,000, preferably 20 to 700, more preferably 30 to 400, even more preferably 30 to 200, and particularly preferably 40 to 100. The number-average molecular weight (Mn) of the polyethylene glycol segment is, for example, 200 to 500,000, preferably 500 to 30,000, more preferably 1,000 to 10,000, even more preferably 1,000 to 7,000, still more preferably 1,000 to 6,000, and particularly preferably 1,000 to 3,000. According to the present specification, the number-average molecular weight is a number-average molecular weight measured by gel permeation chromatography (GPC) and calculated relative to polystyrene standards.

One end of the polyethylene glycol segment is either directly linked to a hydrophobic polyester segment, which will be described below, or is linked to a hydrophobic polyester segment through a linking group. The other end is not particularly limited and may be a hydroxy group at an end of polyethylene glycol or may be any terminal group obtained by modifying a hydroxy group at an end. Examples of the terminal group at the other end include a hydrogen atom, a hydroxy group, an alkoxy group having 1 to 12 carbon atoms which may have a substituent, an alkenyloxy group having 1 to 12 carbon atoms which may have a substituent, and an aralkyloxy group having 7 to 20 carbon atoms which may have a substituent. Examples of the substituent for the alkoxy group having 1 to 12 carbon atoms, the alkenyloxy group having 1 to 12 carbon atoms, and the aralkyloxy group having 7 to 20 carbon atoms include a hydroxy group, an amino group, a formyl group, and a carboxy group. The terminal group at the other end is preferably an alkoxy group having 1 to 6 carbon atoms which may have a substituent, more preferably an alkoxy group having 1 to 6 carbon atoms having no substituent, even more preferably an alkoxy group having 1 to 3 carbon atoms having no substituent, and still more preferably a methoxy group.

Furthermore, the polyethylene glycol segment may have a targeting molecule through the terminal group. Examples of the targeting molecule include a sugar, a lipid, a peptide, a protein, derivatives of those, and folic acid. Furthermore, from the viewpoint of interacting with various proteins present on the surface of nerve cells of the spinal cord and thereby being efficiently deliverable with high specificity to the organ, examples of the targeting molecule include a ligand of a receptor, an antibody, a peptide or protein of a fragment of the ligand or antibody.

### Hydrophobic Polyester Segment

A hydrophobic polyester segment is a hydrophobic segment obtained by polycondensing a monomer having a carboxy group and a hydroxy group in the molecule. The hydrophobic polyester segment may be a homopolymer of a single monomer or may be a copolymer of two or more kinds of monomers. The hydrophobic polyester segment is preferably a homopolymer of a single monomer. Examples of the hydrophobic polyester as a homopolymer include poly(ε-caprolactone) and polylactic acid. Examples of the hydrophobic polyester as a copolymer of two or more kinds of monomers include poly(lactic acid-glycolic acid copolymer). Above all, poly(ε-caprolactone) is preferred as the hydrophobic polyester segment. Regarding the lactic acid moiety of the polylactic acid and the lactic acid-glycolic acid copolymer, any of a D-form, an L-form, or a mixture of the D-form and the L-form may be used; however, a mixture of the D-form and the L-form is preferable.

One end of the hydrophobic polyester segment is either directly linked to the above-mentioned polyethylene glycol segment or is linked to the polyethylene glycol segment through a linking group. The other end is not particularly limited and may be a carboxy group at an end of the hydrophobic polyester segment or may be any terminal group obtained by modifying a carboxy group at an end. Furthermore, a membrane-permeable peptide that will be described later may be linked to the other end directly or through a linking group.

The number-average molecular weight (Mn) of the hydrophobic polyester segment is, for example, 500 to 30,000, preferably 1,000 to 10,000, more preferably 1,000 to 8,000, even more preferably 1,000 to 7,000, and still more preferably 1,000 to 3,000.

The polyethylene glycol segment and the hydrophobic polyester segment in the block type copolymer may be linked directly or indirectly through a suitable linking group; however, preferably the segments are directly linked. The linking mode in which the polyethylene glycol segment and the hydrophobic polyester segment are directly linked is preferably an ester bond formed by a terminal hydroxy group of the polyethylene glycol segment and a terminal carboxy group of the hydrophobic polyester segment. The linking group in the case where the polyethylene glycol segment and the hydrophobic polyester segment are indirectly linked is not particularly limited as long as it is a group that links the two polymer segments by a chemical bond, and any linking group formed from a functional group capable of bonding to the terminal group of the polyethylene glycol segment and the terminal group of the hydrophobic polyester segment may be used. The linking group is preferably an alkylene group having 1 to 6 carbon atoms. The linking mode of the linking group to the polyethylene glycol segment is preferably an ether bond by the terminal oxygen atom of a poly(oxyethylene) group, and the linking mode to the hydrophobic polyester segment is preferably an amide bond or an ester bond.

Specific examples of the block copolymer include a monomethoxy polyethylene glycol-poly(ε-caprolactone) copolymer, a monomethoxy polyethylene glycol-polylactic acid copolymer, and a monomethoxy polyethylene glycol-poly(lactic acid-glycolic acid copolymer) copolymer. Preferred examples include a monomethoxy polyethylene glycol-poly(ε-caprolactone) copolymer. Above all, a monomethoxy polyethylene glycol-poly(ε-caprolactone) copolymer in which the number-average molecular weight of polyethylene glycol is 1,000 to 6,000 and the number-average molecular weight of poly(ε-caprolactone) is 1,000 to 6,000 is preferred, and a monomethoxy polyethylene glycol-poly(ε-caprolactone) copolymer in which the number-average molecular weight of polyethylene glycol is 1,000 to 3,000 and the number-average molecular weight of poly(ε-caprolactone) is 1,000 to 3,000 is more preferred.

The method for producing the block copolymer is not particularly limited, and the block copolymer can be produced by any known method. For example, the block copolymer can be produced by a method of linking a polyethylene glycol segment and a hydrophobic polyester segment by a suitable linking mode. Furthermore, the block copolymer may be prepared by using a terminal hydroxy group of a polyethylene glycol segment as an initiation point and sequentially performing polymerization reactions by ring-opening polymerization with a cyclic ester monomer. Preferably, the block copolymer is prepared by using a terminal hydroxy group of a polyethylene glycol segment as an initiation point and sequentially performing polymerization reactions by ring-opening polymerization with a cyclic ester monomer. By changing the feed ratio of the cyclic ester monomer with respect to the polyethylene glycol segment, it is possible to obtain copolymers having various degrees of polymerization of each of the units. Polyethylene glycol-poly(ε-caprolactone) is produced by using ε-caprolactone as the cyclic ester monomer, and polyethylene glycol-polylactic acid is produced by using dilactide. Polyethylene glycol-poly(lactic acid-glycolic acid copolymer) is produced by using dilactide and glycolide. Regarding specific production methods, for example, Biomaterials, Vol. 24, pp. 3563-3570 (2003), Biomaterials, Vol. 26, pp. 2121-2128 (2005), and International Journal of Pharmaceutics, Vol. 182, pp. 187-197 (1999) can be referred to.

### Membrane-Permeable Peptide

The drug delivery composition according to the present embodiment contains a membrane-permeable peptide.

The term “membrane-permeable peptide” refers to a peptide that can permeate through the cell membrane or mucosa. As the membrane-permeable peptide, any known one can be used without particular limitation. The amino acid residues constituting the membrane peptide may be natural amino acids or unnatural amino acids, and both the L-form and the D-form can be used without particular limitation.

It is preferable that the membrane-permeable peptide contain arginine. Since arginine has a guanidine unit, arginine imparts membrane permeability to a peptide by interacting with the cell membrane or the organellar membrane of an endosome or the like. The number of arginine residues is preferably 30 to 100%, and more preferably 40 to 100%, with respect to the total number of all peptide residues.

Examples of other amino acid residues constituting the membrane-permeable peptide include hydrocarbon-based amino acids such as lysine, glycine, β-alanine, alanine, leucine, isoleucine, valine, and phenylalanine; cyclic amino acids such as proline and tryptophan; sulfur-based amino acids such as cysteine; acidic amino acids such as aspartic acid and glutamic acid; and basic amino acids such as histidine can be used.

An exemplary example of the number of residues in the membrane-permeable peptide is 4 to 30, and the number of residues is 5 to 20, more preferably 5 to 15, even more preferably 6 to 12, and particularly preferably 8 to 10.

Specific exemplary examples of the membrane-permeable peptide include the following.

Tat:
(SEQ ID NO: 1)
GRKKRRQRRRG,
(SEQ ID NO: 3)
GRKKRRQRRRPPQ,

or the like.

Polyarginine: Rn (n=4 to 12)

Arginine-rich peptide

(SEQ ID NO: 2)
CHHRRRRHHC
(SEQ ID NO: 4)
CHHRR
(SEQ ID NO: 5)
HHRRRRHH
(SEQ ID NO: 6)
HHHHRRRR
(SEQ ID NO: 7)
RRRRHHHH
Penetratin:
(SEQ ID NO: 8)
RQIKIWFQNRRMKWKK
Transportan:
(SEQ ID NO: 9)
GWTLNSAGYLLGKINLKAL
Pep-1:
(SEQ ID NO: 10)
KETWWETWWTEWSQPKKKRKV
pVEC Cadherin:
(SEQ ID NO: 11)
LLIILRRRIRKQAHAHSK

According to an exemplary embodiment, the membrane-permeable peptide is linked to an end of the hydrophobic polyester segment, the end being not bonded to the polyethylene glycol segment. Regarding the bonding between the membrane-permeable peptide and the hydrophobic polyester segment, the two may be directly bonded or may be bonded through a linking group; however, it is preferable that the two be directly bonded. The linking mode between the hydrophobic polyester segment and the membrane-permeable peptide is preferably amide bonding formed by the terminal carboxy group of the hydrophobic polyester segment and the terminal amino group of the membrane-permeable peptide. Alternatively, the linking mode between the hydrophobic polyester segment and the membrane-permeable peptide is preferably ester bonding formed by the terminal hydroxy group of the hydrophobic polyester segment and the terminal carboxy group of the membrane-permeable peptide. The linking group in the case where the hydrophobic polyester segment and the membrane-permeable peptide are indirectly linked is not particularly limited as long as it is a group that links the two by a chemical bond, and the linking group may be any linking group formed from functional groups capable of bonding to the terminal group of the hydrophobic polyester segment and the terminal group of the membrane-permeable peptide. The linking group is preferably an alkylene group having 1 to 6 carbon atoms. The linking mode of the linking group with the hydrophobic polyester segment is preferably amide bonding or ester bonding, and the linking mode with the membrane-permeable peptide is preferably amide bonding or ester bonding.

The bonding between the hydrophobic polyester segment and the membrane-permeable peptide can be carried out by, for example, reacting the block copolymer with the membrane-permeable peptide in the presence of a carbodiimide-based condensing agent.

According to another exemplary embodiment, the membrane-permeable peptide contains a lipid-soluble group either directly or through a linking group. By containing a lipid-soluble group, the hydrophobic interaction of the block copolymer with the hydrophobic polyester segment is increased, and the stability of the drug delivery composition is enhanced.

The lipid-soluble group is not particularly limited as long as it is a group that is lipid-soluble; however, the lipid-soluble group is selected from, for example, an alkyl group having 4 to 30 carbon atoms which may have a substituent, an alkenyl group having 4 to 30 carbon atoms which may have a substituent, and an aralkyl group having 7 to 30 carbon atoms which may have a substituent, and is preferably selected from an alkyl group having 8 to 20 carbon atoms which may have a substituent, an alkenyl group having 8 to 20 carbon atoms which may have a substituent, and an aralkyl group having 8 to 20 carbon atoms which may have a substituent. Furthermore, as another embodiment, the lipid-soluble group is preferably selected from a group derived from cholesterol and a group derived from a lipid-soluble vitamin.

Examples of the substituent carried by the alkyl group, alkenyl group, and aralkyl group for the lipid-soluble group include a sulfanyl group, a hydroxy group, an amino group, a halogen atom, a nitro group, a cyano group, a carboxy group, a carbamoyl group, a sulfamoyl group, a carbocyclic aryl group, a heterocyclic aryl group, an alkylthio group, an aralkylthio group, an arylthio group, an alkylsulfinyl group, an aralkylsulfinyl group, an arylsulfinyl group, an alkylsulfonyl group, an aralkylsulfonyl group, an arylsulfonyl group, a sulfamoyl group having a substituent, an alkoxy group, an aralkyloxy group, an aryloxy group, an acyloxy group, an alkoxycarbonyloxy group, an aralkyloxycarbonyloxy group, a carbamoyloxy group having a substituent, a monoalkylamino group, a dialkylamino group, a cyclic amino group, an acylamino group, an alkoxycarbonylamino group, an aralkyloxycarbonylamino group, a ureido group having a substituent, an alkylsulfonylamino group, an arylsulfonylamino group, a sulfamoylamino group having a substituent, an acyl group, an alkoxycarbonyl group, an aralkyloxycarbonyl group, a carbamoyl group having a substituent, and a silyl group. Here, the carbocyclic aryl group, heterocyclic aryl group, alkylthio group, arylthio group, alkylsulfinyl group, arylsulfinyl group, alkylsulfonyl group, arylsulfonyl group, sulfamoyl group having a substituent, alkoxy group, aryloxy group, acyloxy group, alkoxycarbonyloxy group, carbamoyloxy group having a substituent, monoalkylamino group, dialkylamino group, cyclic amino group, acylamino group, alkoxycarbonylamino group, ureido group having a substituent, alkylsulfonylamino group, arylsulfonylamino group, sulfamoylamino group having a substituent, acyl group, alkoxycarbonyl group, carbamoyl group having a substituent, silyl group, and the like may be substituted with a halogen atom, a nitro group, a cyano group, an alkoxy group having 1 to 8 carbon atoms, an aralkyloxy group having 7 to 8 carbon atoms, or the like.

For example, the alkoxy group may be an alkoxy group having 1 to 8 carbon atoms.

For example, the alkoxy group substituted with a halogen atom may be an alkoxy group having 1 to 8 carbon atoms substituted with a halogen atom, and specific examples thereof include a trifluoromethoxy group and a 2,2,2-trifluoroethoxy group.

For example, the alkoxycarbonyloxy group substituted with a halogen atom may be an alkoxycarbonyloxy group having 2 to 9 carbon atoms substituted with a halogen atom, and specific examples thereof include a trifluoromethoxycarbonyloxy group.

The lipid-soluble group is more preferably an alkyl group having 15 to 20 carbon atoms, even more preferably an alkyl group having 15 to 20 carbon atoms, still more preferably a heptadecyl group (stearyl group) or an octadecyl group, and particularly preferably a heptadecyl group (stearyl group).

Examples of the lipid-soluble vitamin for the lipid-soluble group include vitamin A, vitamin D, vitamin E, and vitamin K.

The lipid-soluble group is bonded to the N-terminal amino group or the C-terminal carboxy group of the peptide, directly or through a linking group. When the lipid-soluble group is an alkyl group having 4 to 30 carbon atoms which may have a substituent, an alkenyl group having 4 to 30 carbon atoms which may have a substituent, or an aralkyl group having 7 to 30 carbon atoms which may have a substituent, and the alkyl group, alkenyl group, or aralkyl group is directly bonded to the N-terminal of the peptide, the N-terminal amino group and a carbon atom of the alkyl group, the alkenyl group, or the aralkyl group are directly bonded. However, an embodiment in which the alkyl group, the alkenyl group, or the aralkyl group is bonded to the N-terminal amino group through a suitable linking group is preferable in view of the ease of preparation.

Examples of the suitable linking group include —CO—, —O—CO—, —NH—CO—, —NH—(CH₂)₆₀ —CO——NH—(CH₂)_{α}—NHCO—, —NH—(CH₂)_{α}—OCO—, —O—(CH₂)_{α}—CO—, —O—(CH₂)_{α}—NHCO—, —O—(CH₂)_{α}—OCO—, and —NH—(CH₂)₂—SS—(CH₂)₂—NHCO—. Here, α is an integer of 1 to 12, preferably an integer of 4 to 12, and more preferably an integer of 6 to 12.

The suitable linking group is particularly preferably —CO—.

When the lipid-soluble group is an alkyl group having 4 to 30 carbon atoms which may have a substituent, an alkenyl group having 4 to 30 carbon atoms which may have a substituent, or an aralkyl group having 7 to 30 carbon atoms which may have a substituent, and the alkyl group, the alkenyl group, or the aralkyl group is directly bonded to the peptide C-terminal, the alkyl group, the alkenyl group, or the aralkyl group is bonded to a ketone type structure in place of the hydroxy group of the C-terminal carboxy group. However, an embodiment in which the alkyl group, the alkenyl group, or the aralkyl group is bonded to the C-terminal carboxy group through a suitable linking group is preferred in view of the ease of preparation.

The suitable linking group is preferably an oxy group, an amino group, or a thio group. When an oxy group (oxygen atom) is used as the linking group, the alkyl group having 4 to 30 carbon atoms which may have a substituent, the alkenyl group having 4 to 30 carbon atoms which may have a substituent, or the aralkyl group having 7 to 30 carbon atoms which may have a substituent, all of which are the lipid-soluble groups, is bonded to the peptide in the mode of ester bonding. When an amino group is used as the linking group, the alkyl group, the alkenyl group, or the aralkyl group is bonded to the peptide in the mode of amide bonding. When a thio group (sulfur atom) is used as the linking group, the alkyl group, the alkenyl group, or the aralkyl group is bonded to the peptide in the mode of thioester bonding.

Examples of other suitable linking groups to the C-terminal carbonyl group include —NH—(CH₂)_{α}—NH—, —NH—(CH₂)_{α}—O—, —O—(CH₂)_{α}—NH—, —O—(CH₂)_{α}—O—, and —NH—(CH₂)₂—SS—(CH₂)₂—NH—. Here, a is an integer of 1 to 12, preferably an integer of 4 to 12, and particularly preferably an integer of 6 to 12.

When the lipid-soluble group is a group derived from cholesterol or a group derived from a lipid-soluble vitamin, it is preferable that the lipid-soluble group be bonded to the moiety obtained by removing a hydrogen atom from a hydroxy group of cholesterol or a lipid-soluble vitamin, and to the moiety obtained by removing a hydroxy group from the peptide C-terminal carboxy group (hereinafter, referred to as C-terminal carbonyl group), in the mode of ester bonding. Alternatively, it is preferable that the lipid-soluble group be bonded to the peptide C-terminal carbonyl group through a linking group such as —(CH₂)_{α}—NH— or —(CH₂)_{α}—O—. Here, α is an integer of 1 to 12, preferably an integer of 4 to 12, and particularly preferably an integer of 6 to 12.

Regarding another embodiment of the case where the lipid-soluble group is a group derived from cholesterol or a group derived from a lipid-soluble vitamin, it is preferable that the lipid-soluble group be bonded to the moiety obtained by removing a hydrogen atom from a hydroxy group of cholesterol or a lipid-soluble vitamin, and to the peptide N-terminal amino group, through a linking group such as —CO—, —(CH₂)_{α}—CO—, —(CH₂)_{α}—NHCO—, —(CH₂)_{α}—OCO—, or —(CH₂)₂—SS—(CH₂)₂—NHCO—. Here, α is an integer of 1 to 12, preferably an integer of 4 to 12, and particularly preferably an integer of 6 to 12.

A peptide in which a lipid-soluble group is directly bonded to the N-terminal can be produced by reacting the terminal amino group of the peptide with a compound corresponding to the lipid-soluble group, the compound having an aldehyde group, a ketone group, a suitable leaving group (a halogen, an alkylsulfonyl group, an arylsulfonyl group, or the like), an epoxy group, or the like under known N-alkylation conditions and the like.

A peptide in which the lipid-soluble group is bonded to the N-terminal amino group of the peptide through a linking group can be produced by reacting the terminal amino group with a compound having a corresponding lipid-soluble group, the compound having a carboxylic acid, an ester, an active ester (N-hydroxysuccinimidized or the like), an acid chloride, an activated carbonic acid diester (4-nitrophenylated carbonic acid diester or the like), an isocyanate, or the like under known N-carbonylation conditions or the like.

A peptide in which the lipid-soluble group is directly bonded to the C-terminal can be produced by converting the terminal carboxylic acid of the peptide into an acid chloride, an acid anhydride, or an ester, and reacting the acid chloride, acid anhydride, or ester with an organometallic compound having a corresponding lipid-soluble group, or the like (for example, a Grignard reagent, an organolithium compound, an organozinc compound, or the like) under known ketonization reaction conditions or the like. A peptide in which the lipid-soluble group is bonded to the C-terminal carboxy group of the peptide through a linking group can be produced by reacting the terminal carboxy group of the peptide with a compound having a corresponding lipid-soluble group, the compound having an amino group, a hydroxy group, or a thiol group, by a known condensation reaction. Furthermore, the terminal carboxy group of the peptide can also be reacted by a known condensation reaction or the like, using a substrate converted into an ester, an active ester (N-hydroxysuccinimidized or the like), an acid chloride, or the like.

Regarding specific reaction conditions of the N-alkylation conditions, N-carbonylation conditions, ketonization reaction conditions, and condensation reaction conditions, for example, {Comprehensive Organic Transformations Second Edition. 1999, John Wiley & Sons, INC.} and the like can be referred to. The peptide of the present invention can be produced by the methods described in these known documents, methods equivalent thereto, or combinations of these methods with conventional methods.

Regarding the content ratio of the block copolymer and the membrane-permeable peptide, the block copolymer is preferably 0.05 to 50 equivalents, more preferably 0.2 to 2.0 equivalents, and particularly preferably 0.5 to 1.5 equivalents, with respect to 1 equivalent of the membrane-permeable peptide.

It is preferable that the block copolymer, the membrane-permeable peptide, and the drug form particles, and the particle size thereof be preferably 100 nm or less, more preferably 50 nm or less, and particularly preferably 30 nm or less. It is considered that the hydrophobic polyester segments of the block copolymer are associated by a hydrophobic interaction to form micelle particles, and the drug is encapsulated in these micelles. In a case where the membrane-permeable peptide has a lipid-soluble group, it is considered that the hydrophobic polyester segments of the block copolymer and the lipid-soluble groups of the membrane-permeable peptide are associated by a hydrophobic interaction to form micelle particles, and the drug is encapsulated in these micelles.

The particle size can be measured by a dynamic light scattering method using a light scattering particle size analyzer (for example, manufactured by Malvern Instruments, Ltd., Zetasizer Nano ZS; Otsuka Electronics Co., Ltd., DLS-7000). The light scattering particle size analyzer can measure the cumulant average particle size and the mass-average particle size. Any light scattering particle size analyzers can be used interchangeably; however, the cumulant average particle size measured with Zetasizer Nano ZS manufactured by Malvern Instruments, Ltd. is preferably used.

The method for producing the drug delivery composition according to the present embodiment is not particularly limited. When the membrane-permeable peptide is bonded to an end of the hydrophobic polyester segment of the block copolymer, the drug delivery composition can be produced by dissolving or dispersing the membrane-permeable peptide-linked block copolymer in an appropriate solvent. Examples of the solvent include water, physiological saline, an isotonic glucose solution, and buffer solutions such as phosphate-buffered saline (PBS) and 4-(2-hydroxyethyl)-1-piperazine ethanesulfonic acid (HEPES).

In a case where a lipid-soluble group is bonded to the membrane-permeable peptide, for example, when a water-soluble organic solvent solution including the block copolymer and an aqueous solvent solution including the membrane-permeable peptide are mixed, and the organic solvent is removed, a complex of the block copolymer and the membrane-permeable peptide is formed, and a drug delivery composition can be produced.

Examples of the water-soluble organic solvent include an alcohol solvent such as methanol, ethanol, n-propanol, isopropyl alcohol, t-butyl alcohol, or ethylene glycol; an ether solvent such as 1,2-dimethoxyethane, tetrahydrofuran, or 1,4-dioxane; a ketone solvent such as acetone; a nitrile solvent such as acetonitrile; an amide solvent such as N,N-dimethylformamide or N,N-dimethylacetamide; and a sulfoxide solvent such as dimethyl sulfoxide. Preferably, an ether solvent is used. Above all, it is preferable to use one or more solvents selected from tetrahydrofuran, acetone, acetonitrile, methanol, ethanol, and dimethyl sulfoxide, and it is more preferable to use tetrahydrofuran.

Examples of the aqueous solvent include water, physiological saline, an aqueous glucose solution, and buffer solutions such as phosphate-buffered saline [PBS] and 4-(2-hydroxyethyl)-1-piperazine ethanesulfonic acid [HEPES].

Examples of the method for removing the organic solvent from the mixed solution of the block copolymer and the membrane-permeable peptide include a method using an ultrafiltration membrane (for example, dialysis or a method using a centrifugal ultrafiltration device) and a solvent distillation method; however, a method using an ultrafiltration membrane is preferred.

The pH of a solution of each component and a mixed liquid of those solutions can be appropriately adjusted to the extent that the particle-forming ability is not impaired. The pH is preferably 5 to 9, more preferably 6.5 to 8.0, and even more preferably 7.0 to 8.0. Adjustment of the pH can be easily carried out by using a buffer solution as a solvent. The salt concentration of the buffer solution in a solution of each component and in a mixed liquid of those solutions can be appropriately adjusted as long as the particle-forming ability is not impaired; however, the salt concentration is preferably 1 mM to 300 mM, and more preferably 5 mM to 150 mM.

In the above-described preparation method, it is preferable that the temperature at the time of preparing a solution of each component and at the time of mixing those solutions be set in consideration of the solubility of the block copolymer, and the temperature be usually 0° C. or higher, preferably 0° C. to 60° C., and more preferably 5° C. to 40° C.

In the above-described preparation method, a time for attaining equilibration by leaving the mixed liquid to stand still is provided. Specifically, for example, it is preferable to leave the mixed liquid to stand at 0° C. to 60° C. for 0.1 to 50 hours.

The drug delivery composition according to the present embodiment may contain other components in addition to the above-described components. Regarding the other components, for example, a pharmaceutically acceptable carrier may be mentioned. The phrase “pharmaceutically acceptable carrier” means a carrier that does not inhibit the physiological activity of an active ingredient and does not exhibit substantial toxicity to a target of administration of the active ingredient. The phrase “does not exhibit substantial toxicity” means that at the dosage in which a component is usually used, the component does not exhibit toxicity in the target of administration. Examples of the pharmaceutically acceptable carrier include various additives that are usually used in pharmaceutical products. Examples of the additives include an excipient, an extending agent, a filler, a binder, a wetting agent, a lubricant, a lubricating agent, a surfactant, a disintegrant, a solvent, a solubilizer, a dispersant, a buffer, a stabilizer, a suspending agent, a dissolution aid, a preservative, an antiseptic agent, a corrigent, a soothing agent, an isotonizing agent, a colorant, and a flavoring agent. Regarding such additives, one kind thereof may be used alone, or two or more kinds thereof may be used in combination at any ratio.

The dosage form of the drug delivery composition according to the present embodiment can be any dosage form appropriate for nasal administration. Examples of the dosage form appropriate for nasal administration include a liquid preparation, an aerosol preparation, and a powder preparation.

The drug delivery composition according to the present embodiment includes a kit including the drug delivery composition, the kit being integrally packaged with a package insert describing a method of adding a drug for treatment of a spinal cord disease to the drug delivery composition.

The drug delivery composition according to the present embodiment includes a kit obtained by integrally packaging a first composition containing a block copolymer having a polyethylene glycol segment and a hydrophobic polyester segment linked together, and a second composition containing a cell-permeable peptide. The kit may include a package insert that describes how to mix the first composition with the second composition to produce the above-described drug delivery composition. The content ratio of the block copolymer and the cell-permeable peptide is similar to the content ratio of the drug delivery composition. The block-type copolymer and the peptide may be packed together with the additives and the solvent.

According to the drug delivery composition according to the present embodiment, the drug can be efficiently delivered to the spinal cord by a simple method called nasal administration.

### Pharmaceutical Composition

A second aspect of the present invention is a pharmaceutical composition for treating a spinal cord disease, the pharmaceutical composition containing the drug delivery composition according to the first aspect and a drug for treatment of a spinal cord disease and being administered nasally.

The pharmaceutical composition according to the present embodiment is a pharmaceutical composition for treating a spinal cord disease and contains a drug for treatment of a spinal cord disease. Examples of the spinal cord disease include diseases selected from the group consisting of amyotrophic lateral sclerosis (ALS), spinocerebellar degeneration, spinal muscular atrophy, primary lateral sclerosis, spinobulbar muscular atrophy, chronic pain, spinal cord injury, and spinal cord tumor. Examples of the drug for treating these include drugs similar to those mentioned in the section “[Drug delivery composition]” described above.

A method for producing the pharmaceutical composition according to the present embodiment is not particularly limited. The pharmaceutical composition according to the present embodiment can be produced by, for example, mixing and stirring a solution in which the drug delivery composition according to the first aspect is dissolved or dispersed in an appropriate solvent, and a drug solution in which a drug is dissolved or dispersed in an appropriate solvent. Examples of the solvent include water, physiological saline, an isotonic glucose solution, and buffer solutions such as PBS and HEPES.

The mixing ratio of the drug delivery composition and the drug for treatment of a spinal cord disease may be appropriately set according to the type of the drug. For example, the mixing ratio may be such that drug delivery composition: drug for treatment of a spinal cord disease=1:10 to 10:1 (mass ratio) or the like.

By mixing the drug delivery composition according to the above-described embodiment and a drug, the drug is incorporated into particles (micelles) formed by the block copolymer and the membrane-permeable peptide, and micelles loaded with the drug are formed. By nasally administering the drug in this form, the drug is efficiently delivered to the spinal cord.

The target of application of the pharmaceutical composition according to the present embodiment is preferably an animal that develops a spinal cord disease. For example, the pharmaceutical composition according to the present embodiment can be suitably used for a human being or a mammal other than a human being. The mammal other than a human being is not particularly limited, and examples include primates (monkey, chimpanzee, gorilla, and the like), rodents (mouse, hamster, rat, and the like), rabbit, dog, cat, cow, goat, sheep, and horse.

The dosage form of the pharmaceutical composition according to the present embodiment can be any dosage form appropriate for nasal administration. Examples of the dosage form appropriate for nasal administration include a liquid preparation, an aerosol preparation, and a powder preparation.

The route of administration for the pharmaceutical composition according to the present embodiment is nasal administration. By nasally administering the pharmaceutical composition according to the present embodiment, a drug can be efficiently delivered to the spinal cord.

The pharmaceutical composition according to the present embodiment can administer a therapeutically effective amount of a drug. The term “therapeutically effective amount” means the amount of a drug effective for the treatment or prevention of a target disease. For example, the therapeutically effective amount of a drug may be an amount that can delay the onset and/or progression of a spinal cord disease. The therapeutically effective amount may be appropriately determined according to the type of the drug; the patient's symptoms, body weight, age, gender, and the like; the dosage form of the pharmaceutical composition; the administration method; and the like. For example, regarding the pharmaceutical composition of the present exemplary embodiment, a single dose of the drug can be 0.01 to 1000 mg per 1 kg of the body weight of the target of administration. The dose may be 0.15 to 800 mg/kg, may be 0.5 to 500 mg/kg, may be 1 to 400 mg/kg, or may be 1 to 300 mg/kg.

The pharmaceutical composition according to the present embodiment may include a therapeutically effective amount of a drug per unit dosage form. For example, the content of the drug in the pharmaceutical composition according to the present embodiment may be 0.01% to 90% by mass, may be 0.05% to 80% by mass, or may be 0.1 to 60% by mass.

The administration interval of the pharmaceutical composition according to the present embodiment may be appropriately determined according to the type of the drug; the patient's symptoms, body weight, age, gender, and the like; the dosage form of the pharmaceutical composition; the administration method; and the like. The administration interval can be set to, for example, every few hours, once a day, once every two to three days, or once a week.

### Other Embodiments

According to an exemplary embodiment, the present invention provides the use of a block copolymer having a polyethylene glycol segment and a hydrophobic polyester segment linked together, and a membrane-permeable peptide, for the production of a drug delivery composition for delivering a drug to the spinal cord by nasal administration.

According to an exemplary embodiment, the present invention provides the use of a block copolymer having a polyethylene glycol segment and a hydrophobic polyester segment linked together, a membrane-permeable peptide, and a drug for treatment of a spinal cord disease, for the production of a pharmaceutical composition for treating or preventing a spinal cord disease by nasal administration.

According to an exemplary embodiment, the present invention provides a method for treating a spinal cord disease, the method including nasally administering a pharmaceutical composition including a block copolymer having a polyethylene glycol segment and a hydrophobic polyester segment linked together, a membrane-permeable peptide, and a drug for treatment of a spinal cord disease.

According to an exemplary embodiment, the present invention provides a block copolymer having a polyethylene glycol segment and a hydrophobic polyester segment linked together, and a membrane-permeable peptide, for delivering a drug to the spinal cord by nasal administration.

According to an exemplary embodiment, the present invention provides a block copolymer having a polyethylene glycol segment and a hydrophobic polyester segment linked together, and a membrane-permeable peptide, for treating or preventing a spinal cord disease by nasal administration.

### Examples

Hereinafter, the present invention will be described by way of Examples; however, the present invention is not limited to the following Examples.

### Synthesis Example for PEG-PCL-Tat

The following were used for the synthesis of PEG-PCL-Tat.

MethoxyPEG-PCL (Methoxy poly(ethylene glycol)-block-poly(ε-caprolactone) 2k-2k, Sigma-Aldrich Co.; number-average molecular weight of PCL=2,000, number-average molecular weight of PEG=2,000)

Tat-G (GRKKRRQRRRG(SEQ ID NO: 1), BEX Co., Ltd.)

Tat-G and MPEG-PCL were dissolved in N,N-dimethylformamide (DMF). To this, 1-ethyl-3-(3-dimethylaminopropyl)carbodiimide hydrochloride (WSCI) and 4-dimethylaminopyridine were added, and the mixture was allowed to react at room temperature for 24 hours to form an ester bond between Gly-COOH, which is the C-terminal of Tat-G, and the —OH terminal of MPEG-PCL. The reaction liquid was transferred into a dialysis membrane for an organic solvent (Spectra/Por Dialysis Membranes, MWCO: 3,500), and dialysis was performed against distilled water for 3 days. Subsequently, the reaction liquid was freeze-dried to obtain a powder of Tat-modified MPEG-PCL (PEG-PCL-Tat).

### Example 1

### Nasal Administration Test for RI-Labeled Dextran/PEG-PCL-Tat

### Preparation of RI-Labeled Dextran/PEG-PCL-Tat Complex

[¹⁴C]-Dextran (Mw: 10,000) was used as an RI-labeled dextran. Equal liquid volumes of [¹⁴C]-dextran (4 uCi/mL solvent: HEPES buffer solution (pH 7.4)) and PEG-PCL-Tat (4.8 mg/mL solvent: HEPES buffer solution (pH 7.4)) were mixed, and the mixture was left to stand for 30 minutes to prepare a [¹⁴C]-dextran/PEG-PCL-Tat complex.

### Nasal Administration

Mice (ddY, male, 4 to 6 weeks of age) were administered nasally with the RI-labeled dextran/PEG-PCL-Tat complex. Under inhalation anesthesia using a mask in which the vicinity of the nostrils could be opened or closed, 2 μL each of the above-described complex was alternately administered to the right and left nasal cavities of each mouse every 30 seconds using a micropipette.

### Measurement of Distribution Efficiency

After a predetermined time from the administration of the complex, the olfactory bulb, the forebrain, the hindbrain, and the spinal cord were extracted, and the radioactivity of [¹⁴C] in each of the tissues was measured with a liquid scintillation counter. The RI activity of the administered liquid was also measured in the same manner, and the distribution efficiency (% ID/g tissue) with respect to the dose was calculated.

### Results

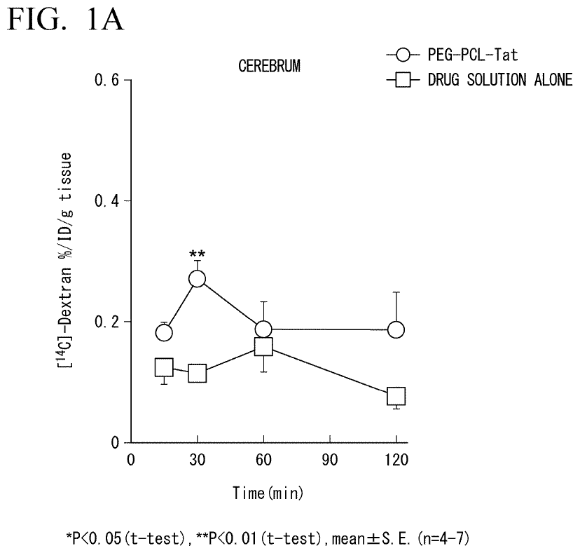

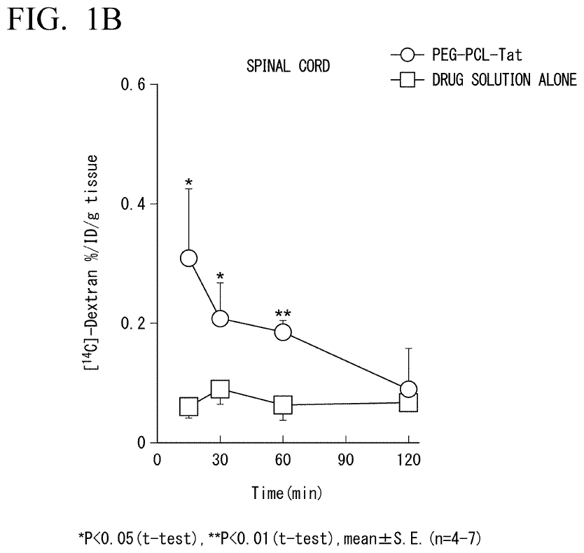

The changes over time in the distribution efficiency of the RI-labeled dextran/PEG-PCL-Tat complex in the cerebrum and the spinal cord are shown in FIG. 1A and FIG. 1B. In FIG. 1A and FIG. 1B, “Drug solution alone” indicates sole administration of RI-labeled dextran, and “PEG-PCL-Tat” indicates administration of the RI-labeled dextran/PEG-PCL-Tat complex. FIG. 1A shows the distribution efficiency in the cerebrum, and FIG. 1B shows the distribution efficiency in the spinal cord. As shown in FIG. 1A and FIG. 1B, in both the cerebrum and the spinal cord, the distribution efficiency was higher in the case where the dextran/PEG-PCL-Tat complex was administered as compared to the case where the drug solution alone (dextran alone) was administered. Particularly, in the spinal cord, the distribution efficiency of the dextran/PEG-PCL-Tat complex became high immediately after administration (FIG. 1B). These results show that the dextran/PEG-PCL-Tat complex is rapidly delivered to the spinal cord after nasal administration.

The distribution efficiency of RI-labeled dextran in each tissue 60 minutes after nasal administration is shown in Table 1. In Table 1, the “Relative ratio” is a relative ratio obtainable when the distribution efficiency in the case of nasally administering the drug solution alone (dextran alone) was taken as 1. In Table 1, “PEG-PCL” represents a dextran/PEG-PCL complex, and “PEG-PCL-Tat” represents a dextran/PEG-PCL-Tat complex.

As shown in Table 1, the distribution efficiency of the dextran/PEG-PCL-Tat complex was the highest in all the tissues.

It was verified that in the spinal cord, the distribution efficiency increased dramatically when the dextran/PEG-PCL-Tat complex was used, as compared to other tissues.

TABLE 1

| | ID %/g tissue | Standard Error | Relative ratio |
|---|---|---|---|
| Olfactory bulb | | | |
| Drug solution alone | 2.104 | 0.501 | 1 |
| PEG-PCL | 1.887 | 0.565 | 0.90 |
| PEG-PCL-Tat | 3.150 | 0.735 | 1.49 |
| Forebrain part | | | |
| Drug solution alone | 0.159 | 0.042 | 1 |
| PEG-PCL | 0.113 | 0.067 | 0.71 |
| PEG-PCL-Tat | 0.187 | 0.046 | 1.17 |
| Hindbrain part | | | |
| Drug solution alone | 0.130 | 0.022 | 1 |
| PEG-PCL | 0.158 | 0.082 | 1.21 |
| PEG-PCL-Tat | 0.317 | 0.052 | 2.43 |
| Whole brain | | | |
| Drug solution alone | 0.177 | 0.033 | 1 |
| PEG-PCL | 0.145 | 0.072 | 0.82 |
| PEG-PCL-Tat | 0.218 | 0.040 | 1.23 |
| Spinal cord | | | |
| Drug solution alone | 0.042 | 0.018 | 1 |
| PEG-PCL | 0.067 | 0.048 | 1.60 |
| PEG-PCL-Tat | 0.185 | 0.016 | 4.40 |

### Reference Example 1

### Oral Administration Test for NAC in ALS Model Mice

### Test Drug

It has been reported that N-Acetyl-Cysteine (NAC) is converted into glutathione (GSH) in cells, suppresses oxidative stress in cells by the antioxidant action of GSH, and exhibits cytoprotective action (Arakawa M and Ito Y. Cerebellum. 2007; 6(4): 308-14.). NAC is considered to be effective for treating ALS as a result of its effect of removing active oxygen. Thus, NAC was used as a test drug for ALS treatment.

### Test Animal

As a test animal, G93A SOD1 transgenic mouse (G93A), which is one of the ALS model animals, was used. This G93A has a mutant SOD1 gene found in familial ALS and is known to exhibit motor paralysis that begins in the lower limbs after 14 to 16 weeks of age (decrease in Rotarod latency, which is one of the indicators of motor function). The research group of the present inventors has already reported the characteristics of the change (progression) in the motor dysfunction of this G93A (Miyagisi H et al., J Pharmacol Sci., 118, 225-236 (2012)).

### Administration of NAC

NAC was orally administered to G93A from 15 weeks of age (105 days of age) after the onset of ALS, and the motor function and the survival time were examined. Water was orally administered as a negative control.

### Evaluation of Motor Function

The motor function was evaluated using Rota-Rod (Muromachi Kikai Co., Ltd.). in order to acclimatize mice to Rota-Rod, mice were trained from 3 weeks (12 weeks of age) before the day of measurement initiation. The rotation speed of Rota-Rod was set to 24 times/minute. The measurement was performed twice a week, and the time (seconds) taken until the mouse fell from Rota-Rod was measured. The cut-off value was set to 300 seconds, while in the case of falling, the average value of three times was used as the score for that day, and measurements were taken up to 133 days after birth.

### Results

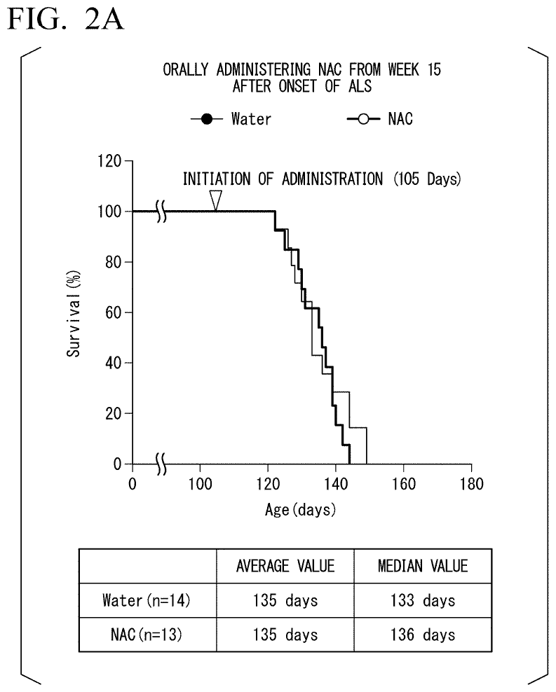

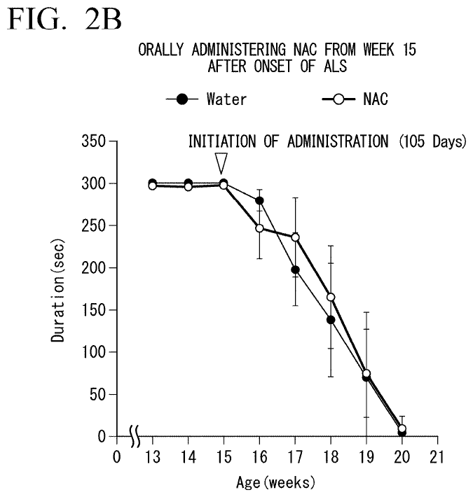

The results are shown in FIG. 2. There was no difference in the (A) motor function and the (B) survival time between the NAC-administered group and the water-administered group, and the effects of suppressing hypanakinesia and prolonging the life span by NAC administration could not be confirmed. Furthermore, the motor function was evaluated using Rota-Rod (Muromachi Kikai Co., Ltd.), and there was no change in the progression of motor dysfunction between the NAC-administered group and the water-administered group.

NAC is known to have low blood-brain barrier (BBB) permeability and low stability in the body (Arakawa M and Ito Y. Cerebellum. 2007; 6(4): 308-14.). The results in FIG. 2 are considered to be due to the fact that since NAC was administered orally, NAC did not migrate to the spinal cord, which is the main lesion of ALS.

### Example 2

### Nasal Administration Test for NAC/PEG-PCL-Tat in ALS Model Mice

### Preparation of Drug Solution

Drug solution of NAC/PEG-PCL-Tat complex:

A drug solution was prepared by mixing equal liquid volumes of NAC dissolved in HEPES buffer solution (pH 7.4) at a concentration of 100 mg/mL and PEG-PCL-Tat dissolved in HEPES buffer solution (pH 7.4) at a concentration of 20 mg/mL and leaving the mixture to stand for 30 minutes.

Drug solution of NAC alone:

A drug solution was prepared by dissolving NAC in HEPES buffer solution (pH 7.4) at a concentration of 50 mg/mL.

### Nasal Administration of Drug Solution

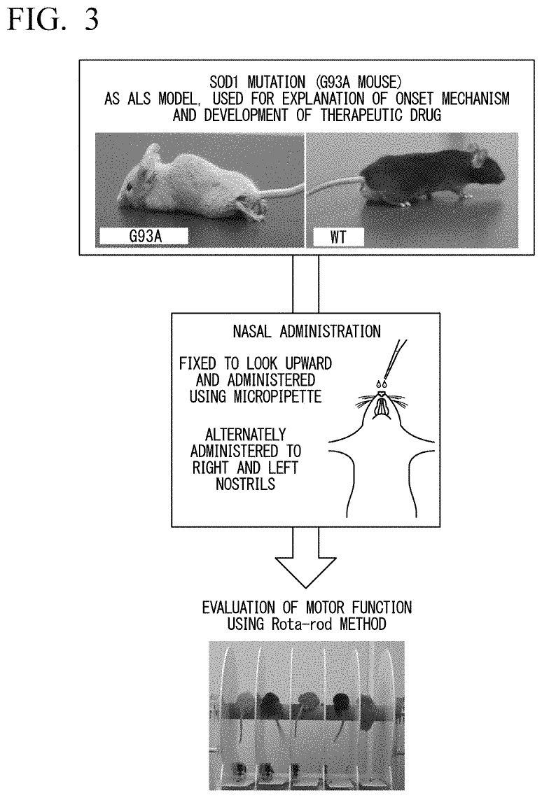

The drug solution was administered to G93A for a period from 105 days of age (15 weeks of age) after birth to 120 days of age, at a frequency of 5 days a week. Nasal administration was performed using a previously reported method (Kanazawa T et al., J Vis Exp., 141, e58485 (2018)). Under inhalation anesthesia using a mask in which the vicinity of the nostrils could be opened or closed, 2 μL each of the drug solution was alternately administered to the right and left nasal cavities every 30 seconds using a micropipette (see FIG. 3). Only male mice were used for the test, and in principle, administration was performed in the morning (10:00 to 12:00). The drug solution-administered group for the NAC/PEG-PCL-Tat complex and the drug solution-administered group for NAC alone consisted of 6 animals each.

### Evaluation of Motor Function

The motor function was evaluated using Rota-Rod (Muromachi Kikai Co., Ltd.). In order to acclimatize mice to Rota-Rod, mice were trained from 3 weeks (12 weeks of age) before the day of measurement initiation. The rotation speed of Rota-Rod was set to 24 times/minute. The measurement was performed twice a week, and the time (seconds) taken until the mouse fell from Rota-Rod was measured. The cut-off value was set to 300 seconds, while in the case of falling, the average value of three times was used as the score for that day, and measurements were taken up to 122 days after birth.

### Measurement of Expression Level of SMI-32

The expression level of SMI-32 was measured by the Western-blot method. Lumbar spinal cord was extracted from mice under deep anesthesia, collected in a cell lysate [150 mM NaCl, 1% Nonidet P-40, 0.5% sodium deoxycholate, 0.1% sodium dodecyl sulfate (SDS), 50 mM Tris-HCl (pH 8.0), 1% Triton X-100, 5 mM EDTA], and homogenized with an ultrasonic homogenizer (Handy sonic, TOMY SEIKO). Subsequently, a supernatant obtained by centrifuging at 6000 g for 15 minutes was used as an extract. Protein quantification was performed by the method of Bradford et al., using bovine serum albumin as the standard substance. Electrophoresis was performed using a 5% to 15% polyacrylamide gel, and then proteins were transferred to IMMOBILON TM-P Transfer Membrane (Millipore). Subsequently, the membrane was blocked using a blocking solution [5% skim milk/Tween Tris-buffered saline (TTBS) [20 mM Tris-HCl (pH 7.6), 137 mM NaCl, 0.05% Tween 20]] for 1 hour at room temperature, and then the proteins were reacted with an anti-SMI32 antibody (1:1000) overnight at 4° C. After washing with TTBS, the proteins were reacted under stirring with an HRP-labeled secondary antibody (1:10000) at room temperature for 1 hour, and after washing, the resultants were detected using ECL or ECL plus (GE Healthcare Life Sciences). β-actin was used as an internal standard. The bands were analyzed using Scion image processing software.

### Results

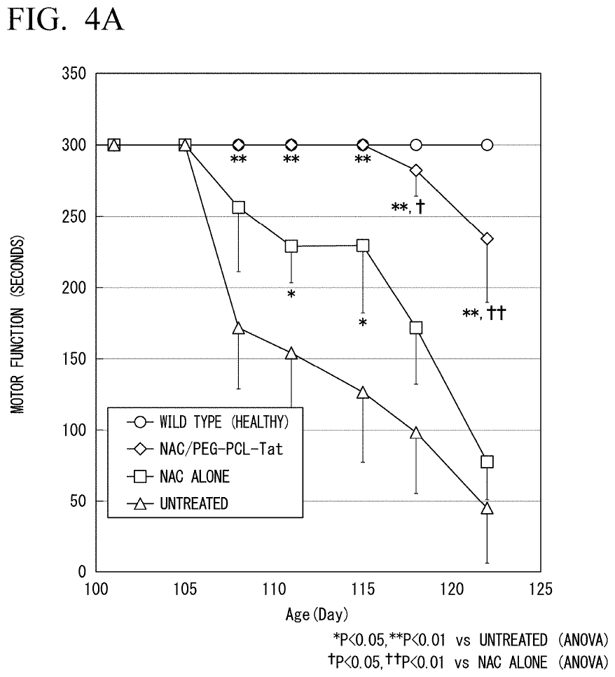

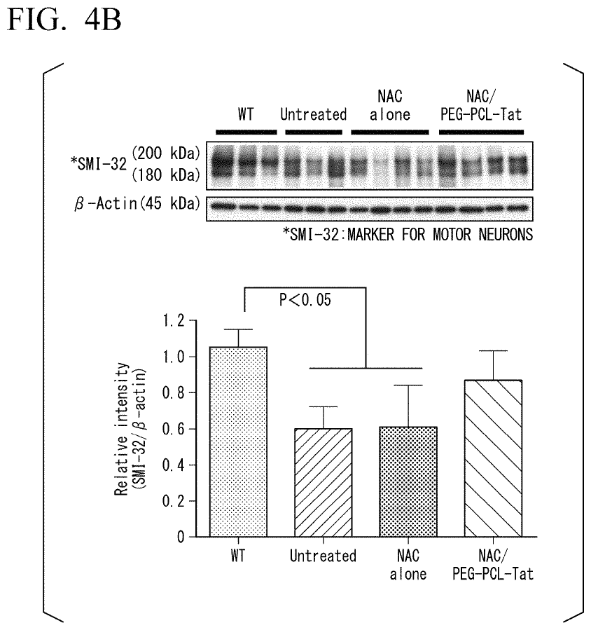

The results are shown in FIG. 4A. In the mice nasally administered with the drug solution of the NAC/PEG-PCL-Tat complex and the drug solution of NAC alone, hypanakinesia was suppressed as compared with the untreated ones. Above all, in the mice nasally administered with the drug solution of the NAC/PEG-PCL-Tat complex, the effect of suppressing hypanakinesia was high.

FIG. 4B shows the results of measuring the expression level of SMI-32, which is a marker for motor neurons, after nasal administration of the drug solution by the Western blot method. The upper diagram of FIG. 4B shows representative band patterns of SMI-32 and β-Actin. The lower diagram of FIG. 4B is a graph showing the values obtained by dividing the band intensity of SMI-32 by the band intensity of β-Actin. In the mice nasally administered with the drug solution of the NAC/PEG-PCL-Tat complex, the expression level of SMI-32 was enhanced as compared with the untreated mice and the mice nasally administered with the drug solution of NAC alone.

These results are considered to be because NAC was delivered to the spinal cord as NAC was administered nasally, and an antioxidant action was exhibited in the spinal cord. Moreover, it is presumed that the NAC/PEG-PCL-Tat complex was highly effective because the distribution efficiency in the spinal cord was enhanced by using the complex.

### Example 3

### Nasal Administration Test for CysA/PEG-PCL-Tat in ALS Model Mice

### Preparation of Drug Solution

Drug solution of CysA/PEG-PCL-Tat complex:

10 mg of PEG-PCL-Tat and 1 mg of cyclosporine A (CysA) were dissolved in methanol, and then the solvent was distilled off by evaporation. The produced thin film of CysA-containing PEG-PCL-Tat was hydrated with HEPES buffer solution (pH 7.4) and then filtered through a 0.8-μm membrane filter, and a drug solution of CysA/PEG-PCL-Tat complex was obtained.

Drug solution of CysA alone:

CysA was dissolved in HEPES buffer solution (pH 7.4) at a concentration of 1 mg/mL, and a drug solution of CysA alone was obtained.

### Nasal Administration of Drug Solution

The drug solution was administered to G93A for a period from 105 days of age (15 weeks of age) after birth to 120 days of age, at a frequency of 5 days a week. Under inhalation anesthesia using a mask in which the vicinity of the nostrils could be opened or closed, 2 μL each of the drug solution was alternately administered to the right and left nasal cavities every 30 seconds using a micropipette (see FIG. 3). Only male mice were used for the test, and in principle, administration was performed in the morning (10:00 to 12:00). The drug solution-administered group for the NAC/PEG-PCL-Tat complex consisted of 8 animals.

### Evaluation of Motor Function

The motor function was evaluated using Rota-Rod (Muromachi Kikai Co., Ltd.). In order to acclimatize mice to Rota-Rod, mice were trained from 3 weeks (12 weeks of age) before the day of measurement initiation. The rotation speed of Rota-Rod was set to 24 times/minute. The measurement was performed twice a week, and the time (seconds) taken until the mouse fell from Rota-Rod was measured. The cut-off value was set to 300 seconds, while in the case of falling, the average value of three times was used as the score for that day, and measurements were taken up to 122 days after birth.

### Results

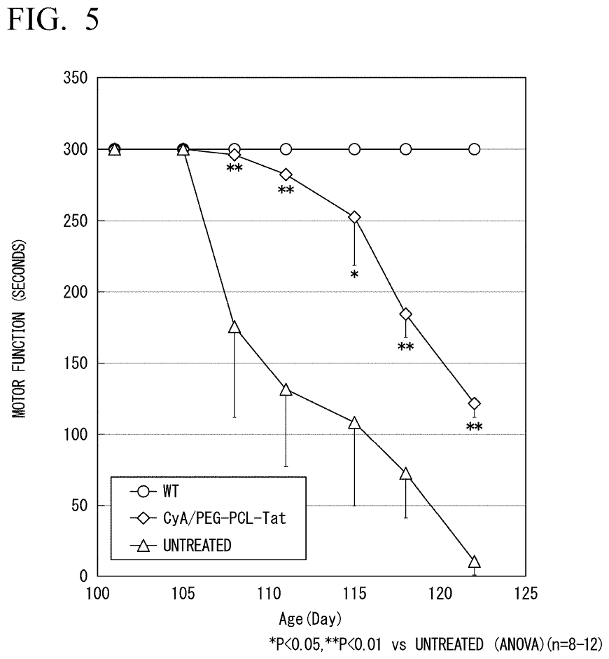

The results are shown in FIG. 5. In the mice nasally administered with the drug solution of the CysA/PEG-PCL-Tat complex, hypanakinesia was suppressed as compared with the untreated ones.

These results are considered to be because CysA was delivered to the spinal cord as the CysA/PEG-PCL-Tat complex was administered nasally, and a therapeutic effect was exhibited for the spinal cord.

### Example 4

### Test for Measuring Relative Expression Level of hSOD1-Targeted siRNA in Spinal Cord by Nasal Administration of Human SOD1 (hSOD1)-Targeted siRNA/PEG-PCL-Tat in ALS Model Mice

(Sequence of siRNA)
hSOD1-targeted siRNA (sense strand sequence):
(SEQ ID NO: 12)
5′-caaagaugcuguggccgaugu-3′
Control siRNA (sense strand sequence):
(SEQ ID NO: 13)
5′-auccgcgcgauaguacguaTT-3′

### Preparation of Drug Solution

Drug solution of hSOD1-targeted siRNA/PEG-PCL-Tat complex:

A drug solution was prepared by mixing equal liquid volumes of hSOD1-targeted siRNA dissolved in HEPES buffer solution (pH 7.4) at a concentration of 160 nmol/mL and PEG-PCL-Tat dissolved in HEPES buffer solution (pH 7.4) at a concentration of 96 mg/mL and leaving the mixture to stand for 30 minutes.

Drug solution of control siRNA/PEG-PCL-Tat complex:

A drug solution was prepared by mixing equal liquid volumes of control siRNA dissolved in HEPES buffer solution (pH 7.4) at a concentration of 160 nmol/mL and PEG-PCL-Tat dissolved in HEPES buffer solution (pH 7.4) at a concentration of 96 mg/mL and leaving the mixture to stand for 30 minutes.

### Nasal Administration of Drug Solution

The drug solution was administered to G93A once a day for 3 consecutive days. Under inhalation anesthesia using a mask in which the vicinity of the nostrils could be opened or closed, 2 μL each of 25 μL of the drug solution in total was alternately administered to the right and left nasal cavities every 30 seconds using a micropipette (see FIG. 3). Only male mice were used for the test, and in principle, administration was performed in the morning (10:00 to 12:00). The number of animals in each drug solution-administered group was four.

### Measurement of Intraspinal Human SOD1 mRNA Expression Level

The mRNA expression level was measured by a real-time PCR method. An RNA extraction reagent was added to extracted spinal cord for homogenization, and then Total RNA was precipitated using 2-propranolol and collected. The collected Total RNA was purified by a DNase treatment. The amount of RNA equivalent to 1 μg was calculated from the concentration calculated from the absorbance, and a reverse transcription reaction was carried out by adding a reagent for reverse transcription to obtain cDNA. A PCR reaction liquid (primer, SYBR Green) was added to the obtained cDNA, and real-time PCR was performed for hSOD1 as the target and GAPDH as the reference. After completion of the reaction, the relative expression level of hSOD1 was calculated by a ΔΔCt method using the number of cycles (Ct value) of hSOD1 and GAPDH.

The method for calculating the relative expression level of hSOD1 is shown below.

(1) The ΔCt value of each spinal cord tissue was obtained from Formula (1).

Ct(hSOD1)−Ct(GAPDH)=ΔCt (1)

Ct: Number of PCR cycles required to reach a certain amount of DNA

(2) ΔΔCt of each spinal cord tissue was determined by Formula (2) by subtracting each ΔCt from the maximum value of ΔCt among all the spinal cord tissues.

ΔCt(max)−ΔCt=−ΔΔCt (2)

(3) The ΔΔCt value was substituted into Formula (3), and the obtained values were averaged for each spinal cord tissue.

2^{-ΔΔCt} (3)

(4) The ratio of each siRNA-administered group/untreated group was determined by Formula (4) as a target mRNA silencing effect.

Target mRNA silencing effect=(2^{-ΔΔCt} of each nucleic acid-administered group)/(average of 2^{-ΔΔCt} of untreated group) (4)

### Results

The results are shown in Table 2. In the mice nasally administered with the drug solution of the hSOD1-targeted siRNA/PEG-PCL-Tat complex, the relative expression level of target hSOD1 mRNA in the spinal cord with respect to GAPDH mRNA was suppressed to 65%, as compared to the control siRNA/PEG-PCL-Tat complex and the untreated ones. These results are considered to be because siRNA was delivered to the spinal cord as the siRNA/PEG-PCL-Tat complex was administered nasally, and an RNA silencing effect was exhibited sequence-specifically in the spinal cord.

TABLE 2

| | Relative hSOD1/GAPDH | Experimental error |
|---|---|---|
| Untreated | mRNA expression ratio | 0.078 |
| hSOD1-targeted | 1 | 0.061 |
| siRNA/PEG-PCL-Tat | | |
| Control | 0.65 | 0.093 |
| siRNA/PEG-PCL-Tat | | |

### Example 5

### Nasal Administration Test for NAC/PEG-PCL-Tat in Neuropathic Pain Model Mice

### Test Animal

As test animals, a rat sciatic nerve semicircle ligation model reported by Seltzer et al. in 1990 (Selzer Z. et al, Pain, 43, 205-218 (1990)) was applied to mice, and partial sciatic nerve ligated mice (PSNL mice) produced by semicircle-ligating the sciatic nerve of the right limb of ICR mice (6 weeks of age, male) with a surgical suture NESCOSUTURE (Alfresa Pharma) under isoflurane (introduction 4%, maintenance 2%) anesthesia were used.

### Preparation of Drug Solution

Drug solution of NAC/PEG-PCL-Tat complex:

A drug solution was prepared by mixing equal liquid volumes of NAC dissolved in HEPES buffer solution (pH 7.4) at a concentration of 100 mg/mL and PEG-PCL-Tat dissolved in HEPES buffer solution (pH 7.4) at a concentration of 25 mg/mL and leaving the mixture to stand for 30 minutes.

### Nasal Administration of Drug Solution

PSNL mice were administered once daily in the morning (10:00 to 12:00) from day 8 to day 13 after nerve ligation. Nasal administration was performed using a previously reported method (Kanazawa T et al., J Vis Exp., 141, e58485 (2018)). Under inhalation anesthesia using a mask in which the vicinity of the nostrils could be opened or closed, 1 μL each of the drug solution was alternately administered to the right and left nasal cavities every 30 seconds using a micropipette (see FIG. 3). Administration of the drug solution of the NAC/PEG-PCL-Tat complex was performed in 10 animals.

### Measurement of Allodynia Response to Tactile Stimuli

Allodynia response to tactile stimuli was measured on day 3, day 5, day 7, day 8, day 9, day 10, day 11, day 12, and day 13 after nerve ligation. Regarding mechanical allodynia, the soles of both feet of a mouse were subjected to von Frey filament (North Coast Medical, Inc.) stimuli, and the minimum pressure at which an escape response appeared was measured by an up-down method (Horiguchi, N et al., Pharmacol. Biochem. Behav., 113, 46-52 (2013)). Before each measurement, the mouse was acclimatized in an acrylic cylinder (diameter 9 cm, height 20 cm) installed on a polypropylene net for at least 1 hour, and the measurement was performed without restraint.

### Results

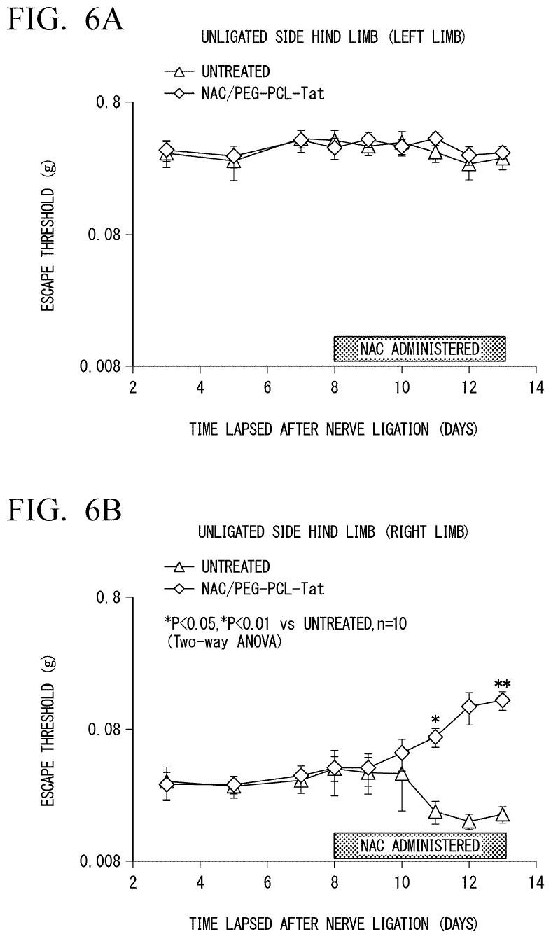

The results are shown in FIG. 6A and FIG. 6B. In mice nasally administered with the drug solution of the NAC/PEG-PCL-Tat complex, the decrease in the escape threshold in the hind limb (right limb) on the nerve ligation side was significantly suppressed as compared to the untreated ones. These results are considered to be because NAC was delivered to the spinal cord as the NAC/PEG-PCL-Tat complex was administered nasally, and a therapeutic effect was exhibited for the spinal cord.

### Preparation Example of PEG-PCL/Peptide Mixed Micelles

The following were used to prepare PEG-PCL/peptide mixed micelles.

MethoxyPEG-PCL (MW: 2000-2000, Sigma-Aldrich Co.)

Stearic acid-modified basic oligopeptide (STR-CHHRRRRHHC, BEX Co., Ltd.; modified peptide in which a stearyl group is bonded to the N-terminal amino group of CHHRRRRHHC (SEQ ID NO:2) through —CO—).

PEG-PCL (9.6 mg) was dissolved in tetrahydrofuran (1.0 mL), and 528 μL was pulled out (Liquid A). The stearic acid-modified basic oligopeptide (4.0 mg) was dissolved in 50 mM HEPES buffer solution (pH=7.4, 1.6 mL), 798 μL was pulled out, and this was mixed with 100 mM dithiothreitol (750 μL) (Liquid B). Liquid A was mixed and stirred with Liquid B, the mixture was diluted with 10 mM HEPES buffer solution (pH=7.4, 1650 μL), and the dilution was concentrated to about 900 μL using a centrifugal filter unit (Amicon Ultra, membrane NMWL 3,000, manufactured by Merck Millipore Corporation). Furthermore, mixed micelles were obtained by repeating dilution and concentration twice with 10 mM HEPES buffer solution (pH=7.4, 1650 μL).

### Example 6

### Nasal Administration Test for RI-Labeled Dextran/PEG-PCL/Peptide Complex

### Preparation of RI-Labeled Dextran/PEG-PCL/Peptide Complex

[¹⁴C]-Dextran (4 μCi/mL solvent: HEPES buffer solution (pH 7.4)) was diluted with 10 mM HEPES buffer solution (pH=7.4, 2250 μL) (Liquid C). Liquid C was mixed and stirred with the PEG-PCL/peptide mixed micelles obtained as described above to obtain a [¹⁴C]-dextran, Mw: 10,000)/PEG-PCL/peptide mixed micelle complex.

### Nasal Administration

Under inhalation anesthesia using a mask in which the vicinity of the nostrils could be opened or closed, 2 μL each of the above-described complex was alternately administered to the right and left nasal cavities every 30 seconds using a micropipette.

### Measurement of Distribution Efficiency

The spinal cord was extracted 30 minutes after administration, and the radioactivity of [¹⁴C] in the spinal cord tissue was measured with a liquid scintillation counter. The RI activity of the administered liquid was also measured in the same manner, and the distribution efficiency (% ID/g tissue) with respect to the dose was calculated.

### Results

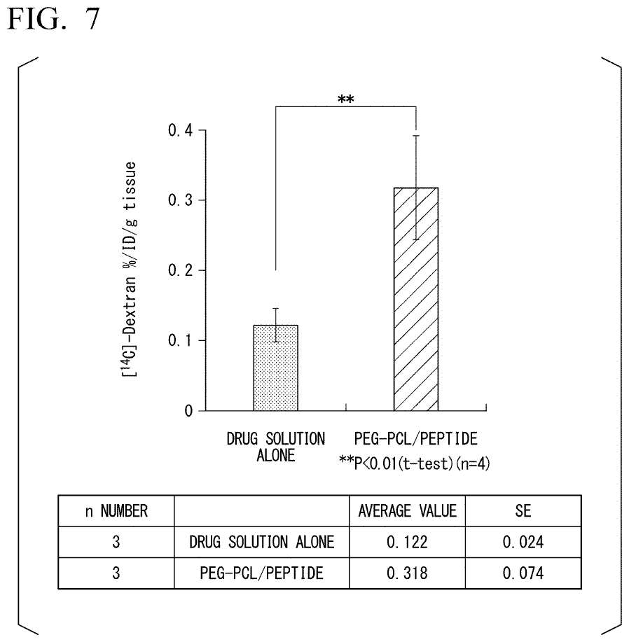

The results are shown in FIG. 7. In FIG. 7, “Drug solution alone” indicates that RI-labeled dextran was administered alone, and “PEG-PCL/peptide” indicates that the RI-labeled dextran/PEG-PCL/peptide complex was administered. As shown in FIG. 7, it was verified that the distribution efficiency of PEG-PCL/peptide in the spinal cord was dramatically enhanced by using a dextran complex.

In Table 3, the distribution efficiencies in the spinal cord obtained when the drug solution alone, the dextran/PEG-PCL complex (PEG-PCL), the RI-labeled dextran/PEG-PCL/peptide complex (PEG-PCL/peptide), or the RI-labeled dextran/PEG-PCL-Tat (PEG-PCL-Tat) was administered nasally are summarized. The distribution efficiency in the spinal cord was highest with the RI-labeled dextran/PEG-PCL-Tat, and the second highest was the RI-labeled dextran/PEG-PCL/peptide complex. From these results, it was verified that PEG-PCL-Tat is most suitable for drug delivery to the spinal cord.

TABLE 3

| | ID %/g tissue | SE | Relative ratio |
|---|---|---|---|
| Drug solution alone | 0.042 | 0.018 | 1 |
| PEG-PCL | 0.067 | 0.048 | 1.60 |
| PEG-PCL/peptide | 0.092 | 0.072 | 2.19 |
| PEG-PCL-Tat | 0.185 | 0.016 | 4.40 |

### Industrial Applicability

According to the present invention, a drug delivery composition capable of delivering a drug to the spinal cord by a simple method with low invasiveness, and a pharmaceutical composition containing the drug delivery composition are provided.

1. A drug delivery composition for delivering a drug to the spinal cord, the drug delivery composition comprising:
a block copolymer having a polyethylene glycol segment and a hydrophobic polyester segment linked together; and
a membrane-permeable peptide of SEQ ID NO:1,
wherein the membrane-permeable peptide is linked to an end of the hydrophobic polyester segment, the end being not linked to the polyethylene glycol segment, and
the drug delivery composition is administered nasally.

2. The drug delivery composition according to claim 1, wherein the drug is a drug for treatment of a spinal cord disease.

3. The drug delivery composition according to claim 2, wherein the spinal cord disease is selected from the group consisting of amyotrophic lateral sclerosis, spinocerebellar degeneration, spinal muscular atrophy, primary lateral sclerosis, spinobulbar muscular atrophy, chronic pain, and spinal cord injury.

4.-6. (canceled).

7. The drug delivery composition according to claim 1, wherein the block copolymer and the membrane-permeable peptide form a micelle.

8. A pharmaceutical composition for treating a spinal cord disease, the pharmaceutical composition comprising:
the drug delivery composition according to claim 1; and
a drug for treatment of a spinal cord disease,
wherein the pharmaceutical composition is administered nasally.

9. The pharmaceutical composition according to claim 8, wherein the spinal cord disease is selected from the group consisting of amyotrophic lateral sclerosis, spinocerebellar degeneration, spinal muscular atrophy, primary lateral sclerosis, spinobulbar muscular atrophy, chronic pain, and spinal cord injury.

10. A method for treating a spinal cord disease, comprising:
nasally administering the pharmaceutical composition according to claim 8.

11. The method according to claim 10, wherein the spinal cord disease is selected from the group consisting of amyotrophic lateral sclerosis, spinocerebellar degeneration, spinal muscular atrophy, primary lateral sclerosis, spinobulbar muscular atrophy, chronic pain, and spinal cord injury.

12. The method according to claim 10, wherein the block copolymer and the membrane-permeable peptide form a micelle.
